(19) United States
(12) Patent Application Publication
(10) Pub. No.: US 2022/0002671 A1
(43) Pub. Date: Jan. 6, 2022

(54) T Cells with Improved Mitochondrial Function

(71) Applicant: Abraham J and Phyllis Katz Cord Blood Foundation, Cleveland, OH (US)

(72) Inventors: Mary Laughlin, Cleveland, OH (US); Jeong Su Do, Cleveland, OH (US)

(21) Appl. No.: 17/291,936

(22) PCT Filed: Nov. 13, 2019
(86) PCT No.: PCT/US2019/061140

§ 371 Date: May 6, 2021

Related U.S. Application Data
(60) Provisional application No. 62/760,392, filed on Nov. 13, 2018.

(51) Int. Cl. C12N 5/0783; A61K 35/17; A61P 37/06

(52) U.S. Cl. CPC C12N 5/0637; A61K 35/17; A61P 37/06; C12N 2501/15; C12N 2502/137; C12N 2533/90; C12N 2535/00; C12N 2506/11

(57) ABSTRACT

Methods for producing therapeutic T cells from umbilical cord blood are provided. Methods for treating immune-related diseases or conditions (e.g. autoimmune diseases, transplant rejection, cancer) using umbilical cord blood derived therapeutic T cells are also provided. Compositions comprising umbilical cord blood derived therapeutic T cells are also provided. Methods for treating diseases and methods for increasing or decreasing available ATP within a proliferating cell, through mitochondrial transfer induction or inhibition are also provided.

### CROSS REFERENCE TO RELATED APPLICATIONS

This application claims the priority benefit of U.S. Provisional Application No. 62/760,392, filed Nov. 13, 2018, which application is incorporated herein by reference.

### FIELD OF THE INVENTION

The present disclosure relates generally to methods for manufacturing T cells and compositions concerning the same. The present disclosure also relates to methods for adoptively transferring T cells to treat an immune-related disease or condition, and compositions comprising the same.

### BACKGROUND

Adoptive cell immunotherapy is an emerging strategy to treat a variety of immune-related diseases and conditions, and involves administering immune system derived cells with the goal of improving immune functionality and characteristics. Adoptive T cell immunotherapy typically requires extracting T cells from a subject, modifying and/or expanding the cells ex vivo, and then introducing the modified and/or expanded T cells into a patient. The application of adoptive cell immunotherapy has been constrained by the ability to isolate, differentiate, modify, and/or expand functional T cells having desired phenotypes and characteristics ex vivo. Therefore, the transition of adoptive T cell immunotherapy from a promising experimental regimen to an established standard of care treatment relies largely on the development of safe, efficient, robust, and cost-effective cell manufacturing protocols. A T cell manufacturing protocol having general applicability is particularly desirable because there are many types of T cell populations, such as inducible regulatory T cells, chimeric antigen receptor-expressing T cells, tumor infiltrating lymphocytes, T cell receptor modified and virus specific effector T cells, which are suitable for use in adoptive T cell immunotherapy.

The present background discusses inducible regulatory T cells as representative example of therapeutic T cells and the need for improved methods of producing therapeutic T cells suitable for use in adoptive cell immunotherapy. There are other types of therapeutic T cells that are suitable for use in adoptive cell immunotherapy, such as chimeric antigen receptor-expressing T cells (CAR-Ts), tumor infiltrating lymphocytes (TIL), and virus specific effector T cells, and these other types of therapeutic T cells can also benefit from improved methods of manufacturing therapeutic T cells and are included in the presently disclosed methods and compositions.

The immune system is finely tuned to efficiently target a broad array of diverse pathogens and keep cancer cells in check, while avoiding reactions against self. To control autoimmunity, humans, similar to all mammals, have developed a number of suppressor cell populations. Among these, regulatory T cells (Tregs) have emerged as the major cell subset maintaining tolerance, with the ability to potently suppress the activation and effector function of other immune cells, including CD4+ and CD8+ T cells, B cells, NK cells, macrophages, and dendritic cells.

Regulatory T cells encompass various subsets of CD4+ and CD8+ cells. In general, these subsets are classified according to their site of development and/or the cytokines they produce. One subset of regulatory T cells develops in the thymus (natural regulatory T cells or “nTreg”) while a different subset develops in the periphery when naïve CD4+ T cells encounter antigen and differentiate into inducible regulatory T cells (“iTregs”) in the presence of TGF-β, IL-10 and IL-2. Both regulatory T cell populations control naïve and ongoing immune responses through a number of independent pathways ranging from direct cell-cell interactions to indirect suppression mediated by soluble cytokines (e.g. IL-10, IL-35 and TGF-β, and metabolic controls. The consequence of these activities is to reduce effector T cell function and promote immune regulation and tolerance.

Since regulatory T cells control pathogenic self-reactive cells, they have therapeutic potential for treating autoimmune diseases as well as suppressing inflammatory conditions (e.g. immune rejection in stem cell, tissue, and organ transplantation, as well as adverse graft vs. host disease). Among the regulatory T cell subsets, CD4⁺ CD25⁺ Foxp3⁺ iTregs offer a promising immunomodulatory treatment strategy due to their role in preventing autoimmunity and enhancing tolerance. The low number of nTregs in human peripheral blood as well as the low proliferative potential of nTregs remain significant challenges to broader clinical applications in adoptive T cell therapy and make them less desirable than iTregs.

Inducible Treg (iTreg) can reestablish tolerance in settings where nTreg are decreased or defective. However, clinical implementation of their potent immune regulatory activity by collection, manufacturing, and dosing quantity and frequency of autologous (self) and allogeneic (other) iTreg in vivo administration has proven challenging. More specifically, experience to date with autologous iTregs has been challenged with the difficulty to expand from the small numbers that can generally be isolated from the peripheral blood, and their functional properties decrease during ex vivo expansion. Moreover, the instability of expression of Forkhead box P3 (FOXP3, a.k.a. FoxP3 and Foxp3) transcription factor that is important for iTreg differentiation and function has to date posed a significant barrier to iTreg clinical application.

FOXP3 is a member of the forkhead/winged-helix family of DNA binding transcription factors and is the master regulator for the development and maintenance of regulatory CD4⁺ 25^{high} Treg. Deletion or mutation of the FOXP3 gene in either mice or in humans can result in severe autoimmune disease, attributable to Treg deficiency. Activated protein 1 (AP-1), Nuclear factor of activated T-cells 1 (NFAT1), Nuclear factor-KB (NF-kB), Small mothers against decapentaplegic 2 (smad2), smad3, and signal transducer and activator of transcription 5 (STATS) all have been identified as regulators of FOXP3 mRNA expression. In addition, stable FOXP3 expression is associated with epigenetic regulatory control in mice.

The regulation of FOXP3 expression in human CD4⁺ T cells is not fully elucidated. Human FOXP3 is expressed by activated CD4⁺ and CD8⁺ T cells as a possible negative feedback loop on cytokine production. In addition, human CD4⁺ T cells have two splice variants of FOXP3 mRNA, while there is only one version in mice. Both human FOXP3 splice variants are co-expressed and no known functional difference has been determined.

Some prior methods have produced autologous iTregs ex vivo by isolating peripheral blood mononuclear cells from blood, stimulating the peripheral blood mononuclear cell population with an antigen to produce iTregs, and recovering and expanding the iTregs. The clinical efficacy of these cells, when transferred to a patient, is hampered by the acquisition of terminal effector differentiation and exhaustion features during expansion ex vivo, thus preventing their function and persistence in vivo. Specifically, large scale ex vivo T cell expansion and effector differentiation can lead to not only robust antigen-specific cytolysis but also to terminal effector differentiation and poor capacity to further expand and persist in vivo. Accumulating evidence suggests that optimal therapeutic effects are achieved when ex vivo generated T cells maintain features associated with early naïve phenotype. Hence, a compromise must be sought to ensure efficient antigen priming while limiting T cell differentiation during the ex vivo culture expansion period.

Therefore, new methods are needed for producing, ex vivo, non-exhausted iTregs that maintain an immature phenotype. New methods are also needed for treating an inflammatory or an autoimmune condition (e.g. autoimmune diseases, transplant rejection, and graft vs. host disease). New iTreg compositions expanded ex vivo in such manner to render sufficient numbers to expectedly have in vivo therapeutic effect whilst maintaining an immature phenotype and lacking exhaustion features are also needed.

More generally, new methods are needed for producing, ex vivo, therapeutic T cells having suitable characteristics (e.g. immature phenotypes, lack of exhaustion features, etc.). New methods are also needed for treating immune-related diseases or conditions with adoptively transferred therapeutic T cells. New therapeutic T cell compositions comprising therapeutic T cells manufactured and/or expanded ex vivo, and which have in vivo therapeutic effect whilst maintaining suitable characteristics (e.g. immature phenotypes, lack of exhaustion features, etc.), are also needed.

### SUMMARY OF THE INVENTION

In embodiments of the invention, methods for manufacturing T cells are provided comprising inducing tunneling nanotube (TNT) transfer of mitochondria from adjacent cells. In embodiments of the invention, the adjacent cells can be a mesenchymal stromal cell (MSC) feeder layer. In embodiments of the invention, mitochondrial transfer can be increased by inducing TNT formation between cells of interest, including T cells. In other embodiments, mitochondrial transfer can be decreased by inhibiting TNT formation between cells of interest, including cancerous cells.

Methods for producing inducible regulatory T cells from blood are provided. In embodiments, blood may be sourced from umbilical cord or adult phlebotomy or pheresis, for example. In certain embodiments, the methods for producing inducible regulatory T cells from blood includes: providing blood; isolating naïve CD4+ T cells from the blood; inducing the naïve CD4+ T cells to differentiate into a first composition comprising iTregs; separating the iTregs from the first composition to form a substantially purified iTreg composition; and expanding the purified iTreg composition over a mesenchymal stromal cell (MSC) feeder layer to form an expanded iTreg composition with sustained FoxP3 expression and suppressive function in inflammatory conditions. In embodiments, the MSCs are induced to form TNT to facilitate mitochondrial transfer to proliferating T cells with sustained FoxP3 expression and suppressive function in inflammatory conditions during ex vivo expansion. In embodiments, the iTregs express CD4+, CD25+, and FoxP3+proteins. In some embodiments the purified iTreg composition is expanded by increasing BACH2 transcriptional regulation of FoxP3 expression.

Methods for treating an inflammatory or an autoimmune condition (e.g. autoimmune diseases, transplant rejection, and graft vs. host disease) using blood derived inducible regulatory T cells expanded over mesenchymal stromal cells with induced TNT formation are also provided. In certain embodiments, the methods for treating an inflammatory or an autoimmune condition in a subject in need thereof includes: administering to the subject a composition comprising a therapeutically effective dose of blood derived iTregs expanded over mesenchymal stromal cells providing mitochondrial transfer.

Compositions comprising umbilical cord blood or adult blood derived inducible regulatory T cells expanded over mesenchymal stromal cells with enhanced mitochondrial transferring TNT activity are also provided.

Methods for producing therapeutic T cells from umbilical cord blood or adult blood are provided. In certain embodiments, the methods for producing therapeutic T cells from umbilical cord blood or adult blood include: providing umbilical cord blood or adult blood; isolating naïve CD4+ T cells from the umbilical cord blood or adult blood; and manufacturing a therapeutic T cell composition from the isolated naïve CD4+ T cells. In certain embodiments, the manufacturing step comprises culturing the therapeutic T cell composition, or a precursor thereto, over a mesenchymal stromal cell (MSC) feeder layer.

In certain embodiments, the methods and manufacturing steps comprise inducing BACH2 transcriptional regulation to increase expression of FoxP3, by methods such as, but not limited to, gene transduction via lentiviral transduction or electroporation.

Methods for treating an immune-related disease or condition are also provided. In certain embodiments, the methods for treating an immune-related disease or condition in a subject in need thereof include: administering to the subject a composition comprising a therapeutically effective dose of a blood derived therapeutic T cell composition, wherein the blood derived therapeutic T cell composition or a precursor thereto was cultured over a mesenchymal stromal cell (MSC) feeder layer with enhanced mitochondrial transferring TNT activity.

Compositions are provided comprising umbilical cord or adult blood derived therapeutic T cells, wherein the umbilical cord or adult blood derived therapeutic T cells or a precursor thereto were cultured over a mesenchymal stromal cell (MSC) feeder layer with induced TNT activity. Methods and compositions are also provided for inducing mitochondrial transfer, such as for the treatment of neurological diseases. Methods and compositions are further provided for inhibiting mitochondrial transfer, such as for the treatment of cancerous tissues.

Methods for increasing available ATP in a cell are provided. In certain embodiments, the methods for increasing ATP in a cell include administering an effective amount of an agent which promotes mitochondrial transfer between a first cell type and a second proliferating cell.

Methods for decreasing available ATP in a cell are provided. In certain embodiments, the methods for decreasing ATP in a cell include administering an effective amount of an agent which prevents/decreases mitochondrial transfer from a first cell to a second proliferating cell.

Methods for treating diseases characterized by either high or low ATP are provided. In certain embodiments, methods for treating diseases characterized by either high or low ATP, include administering an effective amount of an agent that promotes or prevents mitochondrial transfer between cells.

### BRIEF DESCRIPTION OF THE DRAWINGS

FIGS. 1A-1C show MSC mitochondria are transferred into proliferating iTregs during IL-2 driven ex vivo expansion.

FIG. 2 shows UCB iTreg uptake of mitochondria from BM-MSC occurs via tunneling nanotubes during IL-2 driven ex vivo expansion.

FIG. 3 shows UCB iTreg uptake of mitochondria from BM-MSC via tunneling nanotubes during IL-2 driven ex vivo expansion.

FIG. 4 shows UCB iTreg uptake of mitochondria from BM-MSC via tunneling nanotubes during IL-2 driven ex vivo expansion.

FIGS. 5A-5B show quantification of UCB iTreg uptake of mitochondria from MSC during IL-2 driven ex vivo expansion.

FIG. 6 shows that Cytochalasin B blocks mitochondria transfer from BM-MSC into iTregs during IL-2 driven ex vivo expansion.

FIGS. 7A-7B show that Cytochalasin B blocks mitochondria transfer from MSC into UCB iTregs during IL-2 driven ex vivo expansion.

FIGS. 8A-8B show Cytochalasin B treatment significantly inhibits mitochondria transfer to proliferating iTeg during IL-2 driven ex vivo expansion.

FIGS. 9A-9B show that ROS inhibitor does not significantly reduce mitochondria uptake by proliferating UCB iTreg during IL-2 driven ex vivo expansion.

FIGS. 10A-10B show iTregs receiving mitochondria in MSC platform culture have greatly enhanced ROS levels.

FIGS. 11A-11B show mitochondrial membrane potential is enhanced in iTreg IL-2 driven ex vivo expansion conditions over MSC.

FIGS. 12A-12B show that mitochondrial membrane potential is enhanced in iTreg expanded ex vivo in IL-2 over MSC.

FIG. 13 shows iTregs ATP were enhanced in ex vivo expansion conditions over a BM MSC platform.

FIGS. 14A-14H and 15A-15D show that the CD39/CD73 pathway drives MSC mitochondrial transfer into proliferating iTreg.

FIGS. 16A-16B show that CD39/CD73 pharmacological inhibitors block transfer of MSC mitochondria into UCB iTregs during IL-2 driven ex vivo expansion.

FIGS. 17A-17I and 18A-18G show that MSC co-culture with iTregs ameliorates xenogeneic GVHD and allogeneic GVHD in humanized mouse model.

FIGS. 19A-19B show that dysfunctional mitochondria do not transfer into iTregs.

### DETAILED DESCRIPTION

In embodiments of the invention, methods for expanding T cells are provided comprising inducing tunneling nanotube (TNT) transfer of mitochondria from adjacent cells. In embodiments of the invention, the adjacent cells can be a mesenchymal stromal cell (MSC) feeder layer. In embodiments of the invention, mitochondrial transfer can be increased by inducing TNT formation between cells of interest, including T cells. In other embodiments, mitochondrial transfer can be decreased by inhibiting TNT formation between cells of interest, including cancerous cells.

TNT formation and mitochondrial transfer can be induced by compounds such as M-Sec, also known as tumor necrosis factor-a-induced protein, actin polymerization factors including the Rho GTPases family Rac1 and Cdc42, and their downstream effectors WAVE and WASP, and by the expression of the leukocyte specific transcript 1 (LST1) protein in HeLa and HEK cell lines, as described in DuPont et al., Front. Immunol., 25 January 2018 (https://doi.org/10.3389/fimmu.2018.00043). TNT and mitochondrial transfer can also be induced by compounds such as doxorubicin and other anthracycline analogs and other agents that cause cellular stress responses, as described in Desir et al, Scientific Reports, volume 8, Article number: 9484 (2018). TNT and mitochondrial transfer can be inhibited by Cytochalasin B, and nucleoside analogs, such as cytarabine (cytosine arabinoside, AraC), as described in Omsland et al., Scientific Reports, volume 8, Article number: 11118 (2018). Furthermore, Cytochalasin D is cell permeable and an actin inhibitor. Cytocalasin D can cause significant reduction in TNT formation, as shown in Sáenz-de-Santa-María et al., Oncotarget, 2017. See also Hanna et al. Scientific Reports (2017); Keller et al. Invest Ophthalmol Vis Sci. (2017).

Treg express apyrases (CD39) and ecto-5′-nucleotidase (CD73) that promote mitochondrial transfer. CD39/CD73 may be upregulated by using type 1 IFNs, TNFa, IL-1b, prostaglandin (PG) E2, TGF-β, agonists of the wnt signaling pathway, E2F-1, CREB, Sp1, HIF1-a, Stat3, and hypoxia. See Beavis et al., Trends in Immunology (2012); Bao et al., Int'l J. of Molecular Med (2012); Regateiro et al., Eur. J. Immunol (2011); Synnestvedt et al., J. Clin Invest (2002); Eltzschig et al., J of Exp. Med (2003); Eltzschig et al., Blood (2009); and Chalmin et al., Immunity (2012). Gfi-1 represses CD39/CD73 expression, as described in Chalmin, Immunity (2012). CD39/CD73 may also be inhibited using blocking antibodies or pharmacological inhibitors such as POM1 (a E-NTPDases inhibitor), and Adenosine 5′-(α,β-methylene)diphosphate.

Methods for producing inducible regulatory T cells from blood are provided. In embodiments, blood may be obtained from umbilical cord or adult phlebotomy or pheresis, for example. In certain embodiments, the methods for producing inducible regulatory T cells from blood includes: providing blood; isolating naïve CD4+ T cells from the blood; inducing the naïve CD4+ T cells to differentiate into a first composition comprising iTregs; separating the iTregs from the first composition to form a substantially purified iTreg composition; and expanding the purified iTreg composition over a mesenchymal stromal cell (MSC) feeder layer to form an expanded iTreg composition with sustained FoxP3 expression and suppressive function in inflammatory conditions. In embodiments, the MSC are induced to form TNT to facilitate mitochondrial transfer.

Methods for treating an inflammatory or an autoimmune condition (e.g. autoimmune diseases, transplant rejection, and graft vs. host disease) using blood derived inducible regulatory T cells expanded over mesenchymal stromal cells with induced TNT formation are also provided. In certain embodiments, the methods for treating an inflammatory or an autoimmune condition in a subject in need thereof includes: administering to the subject a composition comprising a therapeutically effective dose of blood derived iTregs expanded over mesenchymal stromal cells that provide mitochondrial transfer.

Compositions comprising umbilical cord blood or adult blood derived inducible regulatory T cells expanded over mesenchymal stromal cells with enhanced mitochondrial transferring TNT activity are also provided. In some embodiments, the umbilical cord blood iTregs have been differentiated by inducing BACH2 transcriptional regulation of FoxP3 expression.

Methods for producing therapeutic T cells from umbilical cord blood or adult blood are provided. In certain embodiments, the methods for producing therapeutic T cells from umbilical cord blood or adult blood include: providing umbilical cord blood or adult blood; isolating naïve CD4+ T cells from the umbilical cord blood or adult blood; and manufacturing a therapeutic T cell composition from the isolated naïve CD4+ T cells. In certain embodiments, the manufacturing step comprises culturing the therapeutic T cell composition, or a precursor thereto, over a mesenchymal stromal cell (MSC) feeder layer.

In certain embodiments, the methods and manufacturing steps comprise inducing BACH2 transcriptional regulation to increase expression of FoxP3, by methods such as, but not limited to, gene transduction via lentiviral transduction or electroporation.

Methods for treating an immune-related disease or condition are also provided. In certain embodiments, the methods for treating an immune-related disease or condition in a subject in need thereof include: administering to the subject a composition comprising a therapeutically effective dose of a blood derived T cell composition, wherein the blood derived therapeutic T cell composition or a precursor thereto was cultured over a mesenchymal stromal cell (MSC) feeder layer with enhanced mitochondrial transferring TNT activity.

Compositions are provided comprising umbilical cord or adult blood derived therapeutic T cells, wherein the umbilical cord or adult blood derived T cells or a precursor thereto were cultured over a mesenchymal stromal cell (MSC) feeder layer with induced TNT activity. Methods and compositions are also provided for inhibiting mitochondrial transfer, such for the treatment of cancerous tissues.

Methods for increasing available ATP in a cell are provided. In certain embodiments, the methods for increasing ATP in a cell include administering an effective amount of an agent which promotes mitochondrial transfer between a first cell and a second proliferating cell. In some embodiments, mitochondrial transfer may be promoted with hypoxia. In certain embodiments, mitochondrial transfer is increased by promoting the formation of TNT. In certain embodiments, mitochondrial transfer is promoted through the upregulation of CD39 and/or CD73. In some embodiments, mitochondrial transfer may be promoted using: type 1 IFNs, TNFa, IL-1b, prostaglandin (PG) E2, TGF-β, agonists of the wnt signaling pathway, E2F-1, CREB, Sp1, HIF1-a, a Stat3, or any combination thereof. In some embodiments mitochondrial transfer is promoted using: M-Sec, an actin polymerization factor including in the Rho GTPases family Rac 1 and Cdc42, or their downstream effectors WAVE and WASP, leukocyte specific transcript 1 (LST1), doxorubicin or another anthracycline analog, or another agent that causes cellular stress responses.

Certain diseases are characterized by low ATP. For example, injured neurons may uptake mitochondria from surrounding cells, and promotion of this process may be beneficial to neural repair. Therefore, in some embodiments, methods for increasing ATP in a cell may be used in the treatment of diseases including, but not limited to, neurological diseases, immune diseases, or allergic diseases. In other embodiments, ATP may be increased in cells, such as T cells, in culture expansion conditions, to attain sufficient therapeutic cell doses before administering them to a subject. In some embodiments, an agent that affects mitochondrial transfer is administered directly to a subject. In some embodiments the cell type that provides the mitochondria is MSC.

Methods for decreasing available ATP in a cell are provided. In certain embodiments, the methods for decreasing ATP in a cell include administering an effective amount of an agent which prevents mitochondrial transfer between a first cell and a second proliferating cell. In certain embodiments, mitochondrial transfer is decreased by preventing the formation of TNT. In certain embodiments, an actin inhibitor is administered. In certain embodiments, cytochalasin B, cytochalasin D, or a nucleoside analog, such as cytarabine is administered. In certain embodiments, mitochondrial transfer is decreased through downregulation of the CD39 and/or CD73 signaling pathways. In certain embodiments, CD39 and/or CD73 are downregulated using surface blocking antibodies. In certain embodiments, Gfi-1, E-NTPDases inhibitor, or Adenosine 5′-(α,β-methylene)diphosphate is administered.

Certain diseases are characterized by high ATP. For example, cancerous cells may uptake mitochondria from surrounding cells to promote cancerous growth. Therefore, in some embodiments, methods for decreasing ATP in a cell may be used in the treatment of diseases including, but not limited to, cancer.

Overall, a number of different diseases or conditions may be treated through the promotion or prevention of mitochondrial transfer between cells. In some embodiments cells may be grown in culture and then introduced to a subject. In other embodiments, agents that promote or prevent mitochondrial transfer between cells may be administer directly to a subject.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the term “aberrant immune response” refers to inappropriately regulated immune responses that lead to patient symptoms. Aberrant immune responses can include the failure of a subject's immune system to distinguish self from non-self (e.g. autoimmunity), the failure to respond appropriately to foreign antigens, hyperimmune responses to foreign antigens (e.g. allergic disorders), and undesired immune responses to foreign antigens (e.g. immune rejections of cell, tissue, and organ transplants, and graft vs. host disease).

As used herein, the term “antigen” embraces any molecule capable of generating an immune response. In the context of autoimmune disorders, the antigen is a self-antigen.

As used herein, “immune response” embraces a subject's response to foreign or self antigens. The term includes cell mediated, humoral, and inflammatory responses.

As used herein, “inappropriately regulated” embraces the state of being inappropriately induced, inappropriately suppressed, non-responsiveness, undesired induction, undesired suppression, and/or undesired non-responsiveness.

As used herein, “patient” or “subject” means a human or animal subject to be treated.

As used herein, “proliferation” or “expansion” refers to the ability of a cell or population of cells to increase in number.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

As used herein, the term “regulatory T cell” embraces T cells that express the CD4⁺CD25⁺FoxP3⁺ phenotype.

As used herein, “substantially purified,” “substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially purified” or “substantially separated from” a population of second substances has a measurably lower content of second substances as compared to the non-separated mixture of first and second substances. In one aspect, at least 30%, 50%, 60%, 70%, 80%, 90%, 95%, 98%, 99%, or more of the second substance is removed from the first substance.

The terms “suppression,” “inhibition” and “prevention” are used herein in accordance with accepted definitions. “Suppression” results when an ongoing immune response is blocked or significantly reduced as compared with the level of immune response that results absent treatment (e.g., by the iTreg cells disclosed herein). Similarly, “inhibition” refers to blocking the occurrence of an immune response or significantly reducing such response as compared with the level of immune response that results absent treatment (e.g., by the iTreg cells disclosed herein). When administered prophylactically, such blockage may be complete so that no targeted immune response occurs, and completely blocking the immune response before onset is typically referred to as a “prevention.”

As used herein, “therapeutically effective” refers to an amount of cells that is sufficient to treat or ameliorate, or in some manner reduce the symptoms associated with a disease such as an aberrant immune response. When used with reference to a method, the method is sufficiently effective to treat or ameliorate, or in some manner reduce the symptoms associated with a disease such as an aberrant immune response. For example, an effective amount in reference to a disease is that amount which is sufficient to block or prevent its onset; or if disease pathology has begun, to palliate, ameliorate, stabilize, reverse or slow progression of the disease, or otherwise reduce pathological consequences of the disease. In any case, an effective amount may be given in single or divided doses.

As used herein, the term “treatment” embraces at least an amelioration of the symptoms associated with the aberrant immune response in the patient, where amelioration is used in a broad sense to refer to at least a reduction in the magnitude of a parameter, e.g. a symptom associated with the condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

Methods are provided for generating iTregs. In embodiments, the methods comprise one or more of the following steps: providing umbilical cord blood; isolating naïve CD4+ T cells from the umbilical blood; inducing the naïve CD4+ T cells to differentiate into a first composition comprising iTregs; separating the iTregs from the first composition to form a substantially purified iTreg composition; and expanding the purified iTreg composition over a mesenchymal stromal cell (MSC) feeder layer to form an expanded iTreg composition.

In some embodiments, umbilical cord blood can originate from a variety of animal sources including, for example, humans. Thus, some embodiments can include providing human umbilical cord blood.

In some embodiments, naïve CD4+ T cells are separated/isolated from umbilical cord blood. In some embodiments, naïve CD4+ T cells are substantially separated from other cells in umbilical cord blood to form a purified naïve CD4+ T cell composition. Methods for separating/purifying naïve CD4+ T cells from blood are well known in the art. Exemplary techniques can include Ficoll-Paque density gradient separation to isolate viable mononuclear cells from blood using a simple centrifugation procedure, and affinity separation to separate naïve CD4+ T cells from the mononuclear cells. Exemplary affinity separation techniques can include, for example, magnetic separation (e.g. antibody-coated magnetic beads) and fluorescence-activated cell sorting. In one non-limiting example, mononuclear cells can be obtained from umbilical cord blood by gradient density separation using Ficoll. Non-desired cells (i.e. non CD4+ T cells) from the mononuclear cell fraction can be labeled with biotinylated anti-CD45RO antibodies and magnetically separated/depleted using magnetically assisted cell sorting (“MACS”), leaving behind an enriched/purified population of naïve CD4+ T cells. In some embodiments, at least 75%, 85%, 86%, 87%, 88%, 89%, 90%, 91%, 92%, 93%, 94%, 95%, 96%, 97%, 98%, 99%, or more of the cells of the resulting composition are naïveCD4+ T cells. In some embodiments, the purity of naïveCD4+T cells is equal to or greater than 75%, 85%, 86%, 87%, 88%, 89%, 90%, 91%, 92%, 93%, 94%, 95%, 96%, 97%, 98%, 99%, or more.

In some embodiments, the purified population of naïve CD4+ T cells are induced to render a first composition comprising iTregs. The naïve T cells can be stimulated to render iTregs using methods well known in the art. One exemplary technique for stimulating naïve CD4+ T cells to render iTregs includes culturing naïve CD4+ T cells with Dynabeads (anti-CD3, anti-CD28) at a 1:1 ratio in IL-2 (100 U/ml) and TGF-β1 (5 ng/ml). Activated CD4+ T cells can be harvested and washed after a suitable period of time such as, for example, 96 hours of these stimulation methods.

In some embodiments, iTregs are separated/isolated from the first composition comprising iTregs to form a substantially purified iTreg composition. In some embodiments, iTregs are substantially separated from other cells in the first composition comprising iTregs to form a substantially purified iTreg composition. Methods for separating/purifying/enriching iTregs are well known in the art. Exemplary techniques can include affinity separation methods such as magnetic cell sorting (e.g. antibody-coated magnetic beads) and fluorescence-activated cell sorting to separate iTregs from other cells. In one non-limiting example, iTregs are purified using magnetic separation kits. In some embodiments, at least 75%, 85%, 86%, 87%, 88%, 89%, 90%, 91%, 92%, 93%, 94%, 95%, 96%, 97%, 98%, 99%, or more of the cells of the substantially purified iTreg composition are iTregs. In some embodiments, the purity of iTregs is equal to or greater than 75%, 85%, 86%, 87%, 88%, 89%, 90%, 91%, 92%, 93%, 94%, 95%, 96%, 97%, 98%, 99%, or more. In some embodiments, at least 75%, 85%, 86%, 87%, 88%, 89%, 90%, 91%, 92%, 93%, 94%, 95%, 96%, 97%, 98%, 99%, or more of the cells of the substantially purified iTreg composition are CD4⁺CD25⁺Foxp3⁺.

In some embodiments, the purified iTreg composition is expanded over a mesenchymal stromal cell (MSC) feeder layer to form an expanded iTreg composition. Thus, in embodiments, the purified iTreg composition is expanded to produce a larger population of iTregs. The expansion step can use culture techniques and conditions well known in the art. In certain embodiments, the iTregs are expanded by maintaining the cells in culture for about 1 day to about 3 months. In further embodiments, the iTregs are expanded in culture for about 2 days to about 2 months, for about 4 days to about 1 month, for about 5 days to about 20 days, for about 6 days to about 15 days, for about 7 days to about 10 days, and for about 8 days to about 9 days. The mesenchymal stromal cells (MSC) can be derived from any suitable source (e.g. bone marrow, adipose tissue, placental tissue, umbilical cord blood, umbilical cord tissue).

In some embodiments, the cultured iTregs are expanded at least 2-fold, at least 3-fold, 4, 5, 6, 7, 8, 9, 10, 50, 100, 200, 300, 500, or at least 800-fold. In some embodiments, compositions comprising the expanded iTregs contain a clinically relevant number or population of iTreg cells. In some embodiments, compositions include about 10³, about 10⁴, about 10⁵ cells, about 10⁶ cells, about 10⁷ cells, about 10⁸ cells, about 10⁹ cells, about 10¹⁰ cells or more. In some embodiments, the number of cells present in the composition will depend upon the ultimate use for which the composition is intended, e.g., the disease or state or condition, patient condition (e.g., size, weight, health, etc.), and other health-related parameters that a skilled artisan would readily understand. In addition, in some embodiments, the clinically relevant number of cells can be apportioned into multiple infusions that cumulatively equal or exceed the desired administration, e.g., 10⁹ or 10¹⁰ cells.

In embodiments, transcription factor 'broad complex-Tramtrack-Bric-a-brac domain (BTB) and Cap'n′collar (CNC) homology 1, basic leucine zipper transcription factor 2′ (BACH2) is combined with an ex vivo culture of UCB-derived iTregs to enhance iTreg generation by regulation of Foxp3 expression and the suppressive function of UCB-derived iTregs.

The substantially purified iTregs can be used immediately. The substantially purified iTregs can also be frozen at liquid nitrogen temperatures and stored for long periods of time, being thawed and capable of being used. The cells may be stored, for example, in DMSO and/or FCS, in combination with medium, glucose, etc.

Methods are provided for treating an inflammatory or an autoimmune condition in a subject in need thereof. In embodiments, the methods comprise administering to the subject a composition comprising a therapeutically effective dose of umbilical cord blood derived iTregs expanded over mesenchymal stromal cells.

In some embodiments, the compositions of the present disclosure comprising umbilical cord blood derived iTregs expanded over mesenchymal stromal cells are useful for suppression of immune function in a patient. For example, autologous cells may be isolated, expanded and cultured in vitro as described herein, and subsequently administered back to the same patient. In some embodiments, such treatment is useful, for example, to down-regulate harmful T cell responses to self and foreign antigens, and/or to induce long term tolerance.

In some embodiments, a therapeutically effective amount of a composition comprising umbilical cord blood derived iTregs expanded over mesenchymal stromal cells can be administered to the subject with a pharmaceutically acceptable carrier. Administration routes may include any suitable means, including, but not limited to, intravascularly (intravenously or intra-arterially). In some embodiments, a preferred administration route is by IV infusion. In some embodiments, the particular mode of administration selected will depend upon the particular treatment, disease state or condition of the patient, the nature or administration route of other drugs or therapeutics administered to the subject, etc.

In some embodiments, about 10⁵-10¹¹ cells can be administered in a volume of a 5 ml to 1 liter, 50 ml to 250 ml, 50 ml to 150, and typically 100 ml. In some embodiments, the volume will depend upon the disorder treated, the route of administration, the patient's condition, disease state, etc. The cells can be administered in a single dose or in several doses over selected time intervals, e.g., to titrate the dose.

In one aspect, the compositions and methods disclosed herein are directed to modulating an aberrant immune response in a subject, such as an autoimmune disorder or an allergy, by administering the umbilical cord blood derived iTregs expanded over mesenchymal stromal cells with increased mitochondrial transfer as disclosed herein. In some embodiments, the subject is suffering from an autoimmune disorder or an allergic response, and the umbilical cord blood derived iTregs expanded over mesenchymal stromal cells are used to treat the autoimmune disorder or allergic disorder. In some embodiments, the subject is a human afflicted with an autoimmune disorder or allergic disorder.

The umbilical cord blood derived iTregs expanded over mesenchymal stromal cells disclosed herein can be used to treat, alleviate or ameliorate the symptoms of or suppress a wide variety of autoimmune disorders. In some embodiments, the autoimmune disorders including, but are not limited to, Addison's disease, Alopecia universalis, ankylosing spondylitisis, antiphospholipid antibody syndrome, aplastic anemia, asthma, autoimmune hepatitis autoimmune infertility, autoimmune thyroiditis, autoimmune neutropenia, Behcet's disease, bullous pemphigoid, Chagas' disease, cirrhosis, Coeliac disease, colitis, Crohn's disease, Chronic fatigue syndrome, chronic active hepatitis, dense deposit disease, discoid lupus, degenerative heart disease, dermatitis, insulin-dependent diabetes mellitus, dysautonomia, endometriosis, glomerulonephritis, Goodpasture's disease, Graves' disease, graft versus host disease (GVHD), graft rejection in a recipient following solid organ (e.g., heart, liver, kidney, lung), tissue, bone marrow, or stem cell transplantation, Graves' disease, Guillain-Barre syndrome, Hashimoto's disease, hemolytic anemia, Hidradenitis suppurativa, idiopathic thrombocytopenia purpura, inflammatory bowel disease (“IBD”), insulin dependent diabetes mellitus, interstitial cystitis, mixed connective tissue disease, multiple sclerosis (“MS”), myasthenia gravis, neuromyotonia, opsoclonus myoclonus syndrome, optic neuritis, Ord's thyroiditis, pemphigus vulgaris, pernicious anemia, polyarthritis, polymyositis, primary biliary cirrhosis, psoriasis, Reiter's syndrome, rheumatoid arthritis (“RA”), sarcoidosis, scleroderma, Sjogren's syndrome, systemic lupus erythematosus, Takayasu's arteritis, temporal arteritis, thrombocytopenia purpura, ulcerative colitis, vitiligo, vulvodynia, warm autoimmune hemolytic anemia, or Wegener's granulomatosis.

Additionally or alternatively, in some embodiments, the umbilical cord blood derived iTregs expanded over mesenchymal stromal cells disclosed herein can be used to treat, alleviate or ameliorate the symptoms of or suppress a wide variety of immune related diseases or conditions. In some embodiments, the immune related disease or condition includes, without limitation, allergic conjunctivitis, allergic rhinitis, allergic contact dermatitis, anaphylactoid purpura, asthma, erythema elevatum diutinum, erythema marginatum, erythema multiforme, allergic granulomatosis, granuloma annulare, granlocytopenia, hypersensitivity pneumonitis, keratitis, nephrotic syndrome, overlap syndrome, pigeon breeder's disease, pollinosis, idiopathic polyneuritis, urticaria, uveitis, juvenile dermatomyositis, acute disseminated encephalomyelitis (adem), Addison's disease, agammaglobulinemia, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease/Balo concentric sclerosis, Behcet's disease, Berger's disease, Bickerstaffs encephalitis,

Blau syndrome, bullous pemphigoid, cancer, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, crest syndrome, Crohn's disease, Cushing's Syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, epidermolysis bullosa acquisita, erythema nodosum, erythroblastosis fetalis, essential mixed cryoglobulinemia, Evan's syndrome, fibrodysplasia ossificans progressiva, fibrosing alveolitis (idiopathic pulmonary fibrosis), gastritis, gastrointestinal pemphigoid, glomerulonephritis, Goodpasture's syndrome, Graves' disease, Guillain-Barré syndrome (GBS), Hashimoto's encephalopathy, Hashimoto's thyroiditis, Henoch-Schonlein purpura, herpes gestationis (gestational pemphigoid), hidradenitis suppurativa, Hughes-Stovin syndrome, hypogammaglobulinemia, idiopathic inflammatory demyelinating diseases, idiopathic pulmonary fibrosis, idiopathic thrombocytopenic purpura (autoimmune thrombocytopenic purpura), IgA nephropathy, inclusion body myositis, chronic inflammatory demyelinating polyneuropathy, interstitial cystitis, juvenile idiopathic arthritis (juvenile rheumatoid arthritis), Kawasaki's disease, Lambert-Eaton myasthenic syndrome, leukocytoclastic vasculitis, lichen planus, lichen sclerosus, linear IgA disease (lad), Lou Gehrig's disease (Amyotrophic lateral sclerosis), lupoid hepatitis (autoimmune hepatitis), lupus erythematosus, Majeed syndrome, Méniére's disease, microscopic polyangiitis, Miller-Fisher syndrome (Guillain-Barre Syndrome), mixed connective tissue disease, morphea, Mucha-Habermann disease (Pityriasis lichenoides et varioliformis acuta), multiple sclerosis, myasthenia gravis, myositis, narcolepsy, neuromyelitis optica (devic's disease), neuromyotonia, occular cicatricial pemphigoid, opsoclonus myoclonus syndrome, Ord's thyroiditis, palindromic rheumatism, pandas (pediatric autoimmune neuropsychiatric disorders associated with streptococcus), paraneoplastic cerebellar degeneration, paroxysmal nocturnal hemoglobinuria (pnh), Parry Romberg syndrome, Parsonage-Turner syndrome, pars planitis, pemphigus vulgaris, pernicious anaemia, perivenous encephalomyelitis, poems syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatic fever, sarcoidosis, schizophrenia, Schmidt syndrome, Schnitzler syndrome, scleritis, scleroderma, serum sickness, Sjögren's syndrome, spondyloarthropathy, Still's disease (Juvenile Rheumatoid Arthritis), stiff person syndrome, subacute bacterial endocarditis (sbe), Susac's syndrome, Sweet's syndrome, Sydenham chorea see PANDAS, sympathetic ophthalmia, systemic lupus erythematosis, Takayasu's arteritis, temporal arteritis (giant cell arteritis), thrombocytopenia, Tolosa-Hunt syndrome, transverse myelitis, ulcerative colitis, undifferentiated connective tissue disease, undifferentiated spondyloarthropathy, urticarial vasculitis, vasculitis, vitiligo, wegener's granulomatosis, graft versus host disease (GVHD).

In some embodiments, the umbilical cord blood derived iTregs expanded over mesenchymal stromal cells disclosed herein can be used to treat, alleviate or ameliorate the symptoms of or suppress a wide variety of allergic disorders including, but not limited to, allergic conjunctivitis, allergic rhinitis, allergic contact dermatitis, alopecia universalis, anaphylactoid purpura, asthma, atopic dermatitis, dermatitis herpetiformis, erythema elevatum diutinum, erythema marginatum, erythema multiforme; erythema nodosum, allergic granulomatosis, granuloma annulare, granlocytopenia, hypersensitivity pncumonitis, keratitis, neplirotic syndrome, overlap syndrome, pigeon breeder's disease, pollinosis, idiopathic polyneuritis, urticaria, uveitis, juvenile dermatomyositisitis, and vitiligo.

In some embodiments, the umbilical cord blood derived iTregs expanded over mesenchymal stromal cells with induced TNT formation disclosed herein can be introduced into the subject to treat or modulate an autoimmune disorder or allergic disorder. For example, the subject may be afflicted with a disease characterized by having an ongoing or recurring autoimmune reaction or allergic reaction. In some embodiments, the modulating comprises inhibiting the autoimmune reaction or allergic reaction.

In some embodiments, umbilical cord blood derived iTregs expanded over mesenchymal stromal cells disclosed herein can be administered to a subject for immunotherapy, such as, for example, in tumor surveillance, immunosuppression of cancers such as solid tumor cancers (e.g., lung cancer), and the suppression of in vivo alloresponses and autoimmune responses, including but not limited to, graft versus host disease (GVHD).

The subject methods find use in the treatment of a variety of different conditions and transplant situations in which the modulation of an aberrant immune response in a patient is desired. By way of example, but not by way of limitation, in the case of cellular, tissue, or organ transplantation, a composition comprising umbilical cord blood derived iTregs expanded over mesenchymal stromal cells as disclosed herein may be administered during the time of surgery to prevent graft rejection in an organ transplant patient. To keep the cells at the site until completion of the surgical procedure, in some embodiments, it is convenient to administer the cells in a pharmaceutically acceptable carrier, such as an artificial gel, or in clotted plasma, or by utilizing other controlled release mechanism known in the art.

Manipulation of TNT and mitochondrial transfer may be used in the treatment of any number of diseases. For example, by inducing mitochondrial transfer iTregs with improved number and function are produced; these iTregs may be used in the treatment of many diseases such as autoimmune disorders or allergic disorders. In some embodiments, the disclosed methods can be used to either promote or inhibit mitochondrial transfer to non-T cell types. Intercellular mitochondrial transfer by MSC has been previously described in neuronal injury and cancer models (Babenko et al., 2015). For example, proliferating acute leukemia blasts in the marrow microenvironment have been shown to take mitochondria from MSC (Marlein et al., 2017). Therefore, mitochondrial transfer inhibition may be used to treat cancer. Conversely, the promotion of mitochondrial transfer may be used to treat neurological diseases for example.

### EXAMPLES

### Summary

Ex vivo expansion in standard media/IL-2 conditions was observed over a MSC platform which significantly improved UCB iTreg number, phenotype, and function, compared to standard media/IL-2 suspension cultures alone.

To determine potential mechanisms underlying improved iTreg number and function during 3 week IL-2 driven ex vivo expansion over MSC, experiments were performed to determine whether mitochondria may be transferred from MSC to proliferating UCB iTregs during IL-2 driven ex vivo expansion. Experiments demonstrated transfer of MSC mitochondria to proliferating iTregs via tunneling nanotubules (TNT) during IL-2 driven ex vivo expansion.

Use of a human bone marrow mesenchymal stromal cells (hBM-MSC) platform significantly enhanced the number of iTreg during IL-2 driven 21 day ex vivo expansion vs. standard suspension culture condition (MSC platform: 80.2×10⁶ vs. IL2/media: 39.3×10⁶, n=6; p<0.01). Also, the number of iTreg expressing a naive phenotype (CD4⁺CD4RA⁺ and CD4⁺CD62L⁺) were significantly increased (CD45RA⁺; MSC platform: 74.4±1.6×10⁶ vs. IL2/media: 45.9±2.9×10⁶, n=6, p<0.001; CD62L⁺; MSC platform: 79.1 ±1.3×10⁶ vs. IL2/media: 54.5±2.1×10⁶, n=6, p<0.001), as well as stability of Foxp3 expression (IL-2/media: 88.2±1.7% vs. MSC platform: 96.2±1.1%, n=7; p<0.05). In addition, iTreg suppressive function was noted to be more potent during 21 day IL-2 driven ex vivo expansion compared to standard IL-2/media culture condition (MSC platform: 79% vs. media: 35% inhibition of T cell proliferation in 10:1 ratio, n=6; p<0.01). iTreg expanded over a hBM-MSC platform exhibited higher surface CD25, CTLA-4, and ICOS MFI expression (CD25; MSC platform: 1410 vs. Media: 774; p<0.001, CTLA-4; MSC platform: 1084 vs. Media: 318; p<0.001, ICOS; MSC platform: 4386 vs. Media: 2641, p<0.01, n=6). Notably, hBM-MSC enhancement of iTreg ex vivo expansion requires direct cell-cell contact, as Foxp3 expression in iTreg was not enhanced by hBM-MSC conditioned media (CM:73.4 ±6.8% vs. MSC platform: 96.2 ±1.0%, p<0.001; and IL2/media: 88.8 ±1.6% vs. MSC platform: 96.2±1.0%, p<0.01) nor in a trans-well culture experiments (Transwell: 83.4±2.5% vs. IL2/media: 88.8±1.6%; and Transwell: 83.4±2.5% vs. MSC platform: 96.2±1.0%, p<0.01).

Optical sectioning microscopy and flow cytometry revealed that hBM-MSC supports iTreg number and function via direct contact-dependent mitochondrial transfer. Cytochalasin B treatment blocked mitochondrial transfer, suggesting that tunneling nanotubes

(TNT) facilitate mitochondrial transfer from hBM-MSC to iTreg during IL-2 driven ex vivo expansion (Mock: 2208±122.1 vs. Cyto B: 923.8±89 MFI, n=6, p<0.0001). Moreover, the quantity of ATP (n=6; p<0.01) mitochondrial potential of iTreg (MSC platform: 9010±224.5 vs. media: 7316±122.7 MFI, n=6; p<0.01) were significantly enhanced in iTreg during IL-2 driven ex vivo expansion over a hBM-MSC platform. Taken together, hBM-MSC significantly improves the number, maturation, and function of iTreg during 21 day IL-2 driven ex vivo expansion. One key mechanism of action of hBM-MSC underlying these favorable effects on iTreg during ex vivo expansion was identified to be mitochondrial transfer via TNT. Notably, this invention identifies a novel role of hBM-MSC to overcome current limitations in IL-2/media suspension culture conditions including T cell senescence, and loss of Foxp3 expression.

After it was observed that MSC mitochondrial transfer relies on TNT rather than via episomal transfer (Sinclair et al., 2013; Vignais et al., 2017), it was determined it was driven by mitochondrial metabolic function (CD39/CD73 signaling) in proliferating iTreg during short-term (21 day) IL-2 ex vivo expansion. Enhanced expression of BACH2, SENP3 was noted in iTregs co-cultured with MSCs which promoted Foxp3 stability in iTregs expanded in this condition. Mitochondrial metabolic function (CD39/CD73 signaling) in proliferating iTreg was also noted to induce MSC mitochondria Rho-GPTase 1) Miro1 expression. Miro-1 serves to attach mitochondria to the KLF 5 kinesin motor protein to ensure concerted mitochondrial transport (Chang et al., 2011; Quintero et al., 2009).

Together, these studies provide insight into cellular and molecular mechanisms that drive MSC mitochondrial transfer to proliferating iTreg that serve to maintain robust Foxp3 expression and suppressive function despite adverse inflammatory milieu in vitro and in vivo.

### DETAILED DESCRIPTION OF EXAMPLES

### Summary of Experimental Methods

Foxp3+ iTregs induction: Magnetic bead enriched UCB CD4+ T cells (Miltenyi Biotech, Auburn, Calif.) were stimulated with dynabeads (CD2/3/28) at a concentration 5×10⁵ cells/ml in IL-2 (100 U/ml) and TGF-β (5 ng/ml).

Expansion iTregs: iTregs were collected after 4 days differentiation in TGF-β, and set up for ex vivo expansion. Cells were stained with CellTrace Far Red and 5×10⁵ cells/ml seeded with 100 U/ml IL-2 added.

Dye staining: BM-MSCs were resuspended in complete media at 2×106 cells/ml. Cells were incubated 45 min at 37° C. with 200 nM MitoTracker Green FM. iTregs were also resuspended at 2×10e6 cells/ml. Cells were incubated 20 min at 37° C. with 5 uM CellTrace Far Red. BM-MSC 5×10e5 cells/ml were seeded into 6 well plates (9.4 cm2). Analysis: FACS Fortessa.

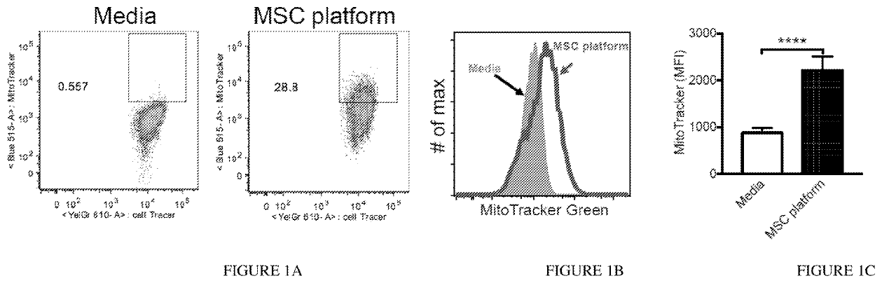

FIG. 1A-1C show BM-MSC were pre-stained with MitoTracker green FM for 30 min and then cultured with iTregs. After 24 h co-culture iTregs were analyzed using MitoTracker green MFI by flow cytometry. CellTrace and CD4+iTreg cells were gated (98.2%) and analyzed for MitoTracker+ iTreg cells. Media condition, MitoTracker+ iTregs expanded in media/IL-2 alone (Media) were 0.557% and in MSC platform expansion condition rendered 28.8% are MitoTracker+ iTregs. These results demonstrate that mitochondria are transferred from BM-MSC into iTregs during IL-2 driven ex vivo culture. Data shown from three different experiments ±SD (n=6). ****, P<0.0001.

UCB iTregs uptake mitochondria from MSC during IL-2 driven ex vivo expansion. Day 0-4 Foxp3+ iTregs induction: UCB CD4+ T cells were stimulated with dynabeads (CD2/3/28) at 5×10⁵ cells/ml in IL-2 (100 U/ml) and TGF-β (5 ng/ml). Expansion iTregs: iTreg cells were collected after 4 days differentiation in TGF-β+2 days rest and used for co-culture over BM-MSC monolayer. Dye labeled iTregs were seeded at 5×10⁵ cells/ml with media/100 U/ml IL-2 added. Dye staining: BM-MSCs were resuspended in complete media at 2×10⁶ cells/ml. Cells were stained with CFSE or PKH and immediately BM-MSCs were incubated 45 min at 37° C. with MitoTracker Red FM (500 nM) or MitoTracker green FM (200 nM). iTregs were also resuspended at 2×10⁶ cells/ml. iTregs were incubated 30 min at 37° C. with lug Hoechst. BM-MSC: 5×10⁵ cells/ml. Microscopy image analysis was performed using ZEN 2012 software (Carl Zeiss).

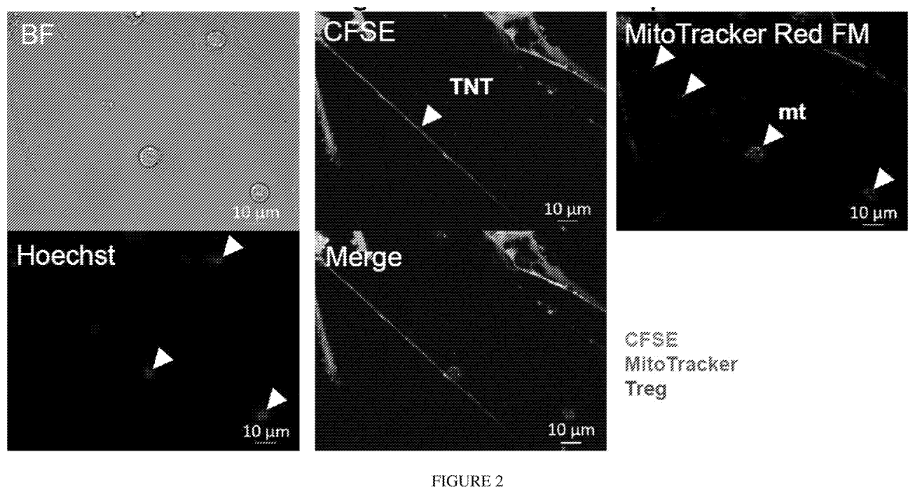

FIG. 2 shows UCB iTreg uptake of mitochondria from BM-MSC occurs via tunneling nanotubes during IL-2 driven ex vivo expansion. After 24 hours, UCB iTregs (Hoechst) were analyzed using MitoTracker Red FM by confocal microscopy. BM-MSC were pre-stained with CFSE and MitoTracker Red. Analysis of recorded images was performed using Zen 2012 (Carl Zeiss) software. This data supports that UCB iTregs receive BM-MSC mitochondria via TNT direct contact. Images are representative from 4 different experiments (n=6).

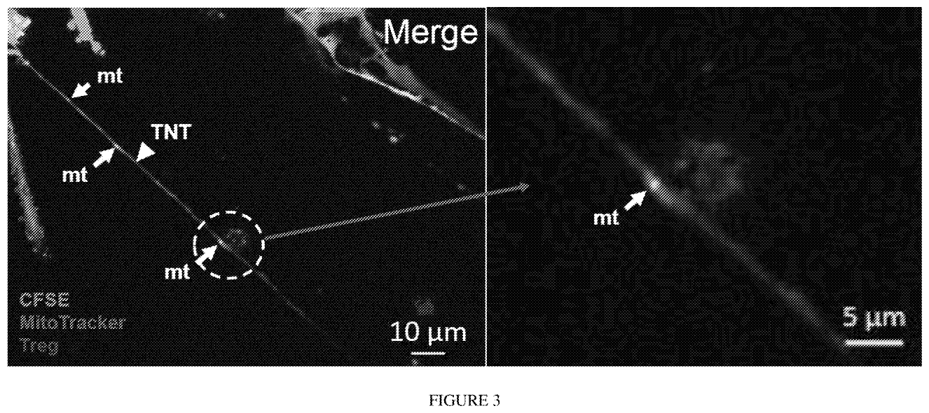

FIG. 3 shows at higher magnification that after 24 h, UCB iTregs were analyzed using MitoTracker Red FM by confocal microscopy. BM-MSC were pre-stained with CFSE and MitoTracker Red FM and then cultured for 24 h. Analysis of recorded images was performed using Zen 2012 (Carl Zeiss) software. iTregs are observed immediately adjacent to MSC TNT which contains mitochondria (red arrow). This data supports that UCB iTregs take up BM-MSC mitochondria via TNT direct contact. Images are representative from 4 different experiments (n=6).

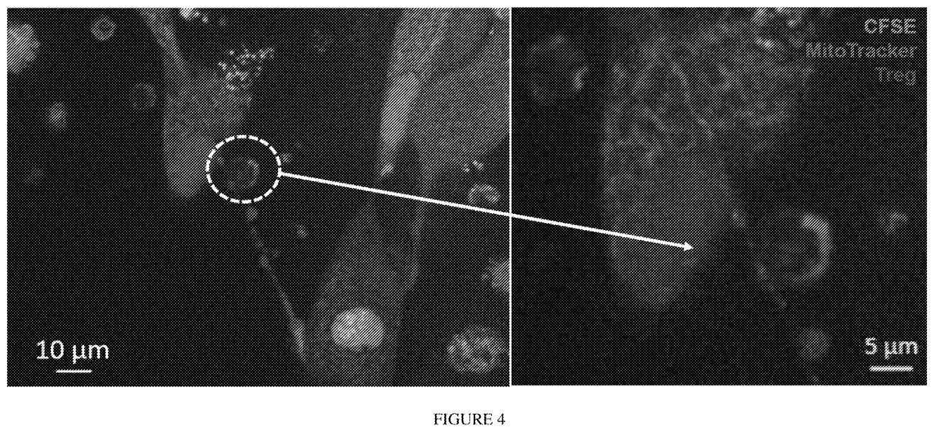

FIG. 4 shows experimental methods are described in slide 18. BM-MSC were pre-stained with CFSE and MitoTracker Red FM and then cultured with iTreg for 24 h. After 24 h, UCB iTregs were analyzed using MitoTracker Red FM by confocal microscopy. Analysis of recorded images was performed using Zen 2012 (Carl Zeiss) software. These images support that UCB iTregs take up MSC mitochondria via TNT direct contact. Images are representative from 4 different experiments (n=6).

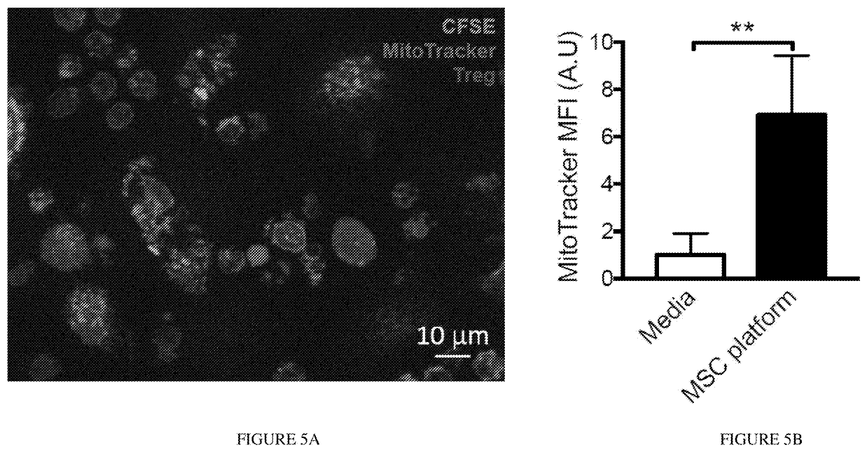

FIGS. 5A-5B show that BM-MSC were pre-stained with CFSE and MitoTracker Red FM and then cultured with iTreg for 24 h. After 24 h, UCB Tregs were analyzed using MitoTracker Red FM by confocal microscopy. Analysis of recorded images was performed using Zen 2012 (Carl Zeiss) software. Here, the image shows that the majority of iTreg are MitoTracker positive, indicating uptake of MSC mitochondria. Results were normalized by iTreg cultured in media/IL-2 alone. Data are representative of three independent experiments±SD (n=6). **, P<0.01.

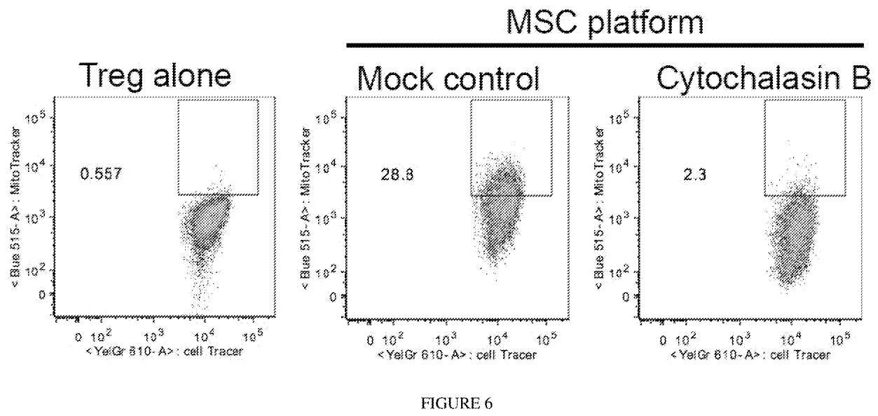

FIG. 6 depicts use of Cytochalasin B, an F-actin-depolymerizing agent known to abolish TNT formation. To test whether mitochondrial transfer occurs via Tunneling NanoTubule (TNT), BM-MSC were treated with Cytochalasin B dissolved in DMSO and compared with DMSO alone for mock control. MSC were pre-stained with MitoTracker green FM for 30 min and then cultured with iTreg (MSC Platform) including mock DMSO control (Mock control) and Cytochalasin B (350 nM) treated BM-MSC (Cytochalasin B) for 24 h, and compared with iTreg alone (Treg alone). After 24 h iTregs were analyzed using MitoTracker green MFI by flow cytometry. Flow data are representative from three different experiments (n=6).

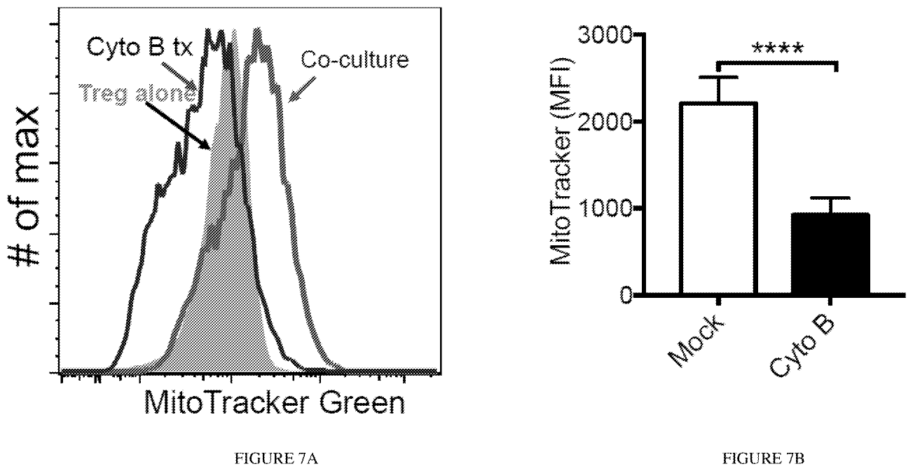

FIGS. 7A-7B show that mitochondrial transfer occurs via Tunneling NanoTubule (TNT). BM-MSC were treated with Cytochalasin B and compared with mock control (Co-culture). MSC were pre-stained with MitoTracker green FM for 30 min and then cultured with iTreg (Co-culture; Red) and compared with iTreg alone (Treg alone; shaded) and Cytochalasin B (350 nM) treated BM-MSC (Cyto B tx; blue) for 24 h. After 24 h co-culture with BM-MSC treated with Cytochalasin B, iTregs were analyzed using MitoTracker green MFI by flow cytometry.

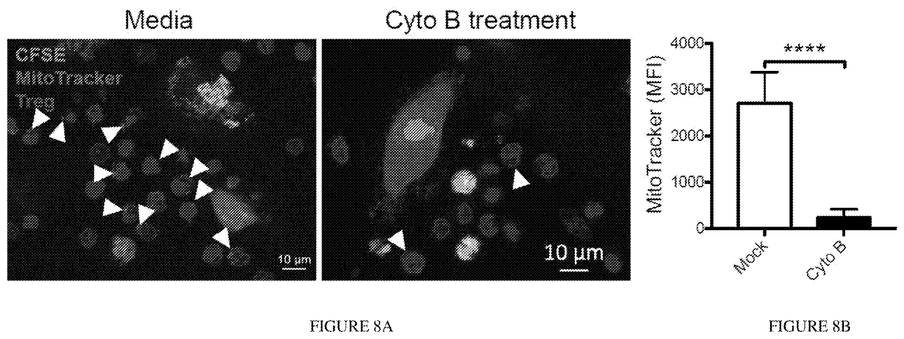

FIGS. 8A-8B show that BM-MSC were pre-stained with CFSE and MitoTracker Red FM and then cultured with UCB iTregs with and without Cytochalasin B (350 nM) for 24 h. After 24 h, UCB iTregs (Hoechst) were analyzed using MitoTracker Red FM by confocal microscopy. Analysis of recorded images was performed using ImageJ or Zen 2012 (Carl Zeiss) software. Arrows show BM- MSC mitochondria within iTreg. Image data shows significantly reduced number of MitoTracker+iTregs detected. Data shown from three different experiments±SD (n=6). ****, P<0.0001. These data demonstrate that Cytochalasin B treatment significantly blocks mitochondrial transfer from MSC to UCB iTregs during IL-2 driven ex vivo expansion. Together, these results support that TNT is a critical conduit for mitochondrial transfer from BM-MSC to UCB iTregs during IL-2 driven ex vivo expansion.

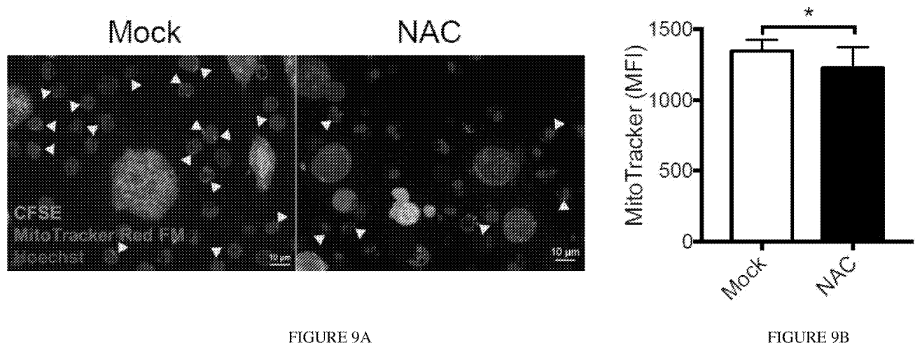

FIGS. 9A-9B show that ROS inhibitor: antioxidant N-acetylcysteine (NAC) 200 uM was added to BM-MSC+iTreg culture for 24-36 h. Data are representative of two independent experiments ±SD (n=6). **, P<0.01. Image was take at 24-36 h incubation. Analysis of recorded images was performed using Zen 2012 (Carl Zeiss) software. Arrows show BM-MSC mitochondria in iTreg. Image results demonstrate that addition of this ROS inhibitor minimally reduced mitochondria uptake by Treg, as there are MitoTracker+iTregs detected. These image results support that mitochondrial transfer from BM-MSC into Tregs is not dependent on ROS mediated mechanism of action. *P<0.05.

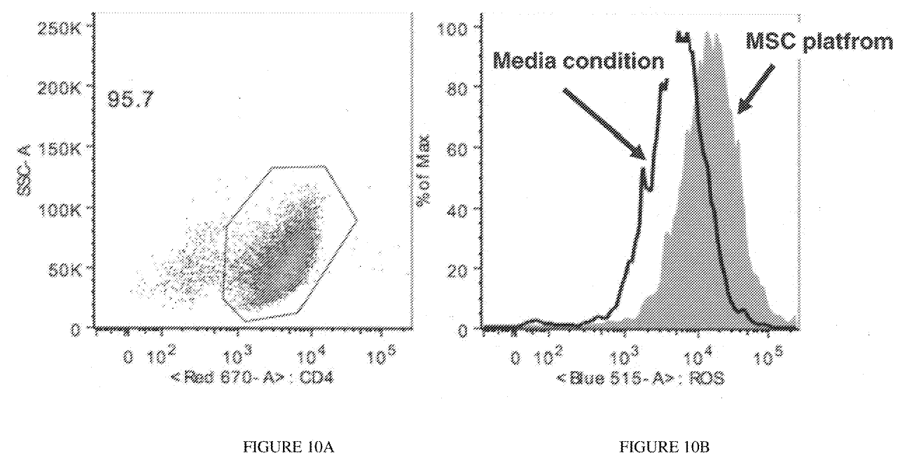

FIGS. 10A-10B show iTregs in MSC platform culture have greatly enhanced ROS levels.

Studies were designed to identify the mechanisms underlying the observation that IL-2 driven ex vivo expansion of iTreg over MSC significantly enhances the number and function of iTregs. Cell expansion: 2-5×10⁵ cells were sub-cultured with ex-vivo media with added IL-2 (100 U/ml). Media was changed every 2-3 days. UCB iTreg cells were harvested at day 21-23 from media/IL-2 alone vs media/IL-2 over a MSC platform. Tetramethylrhodamine (TMRM): Methyl ester is a cell-permeant dye. It accumulates in healthy active mitochondria membrane. Cells were stained with Image-iTTM TMRM (invitrogen) following manufacturers instruction. Cells were analyzed by FACS and confocal microscopy.

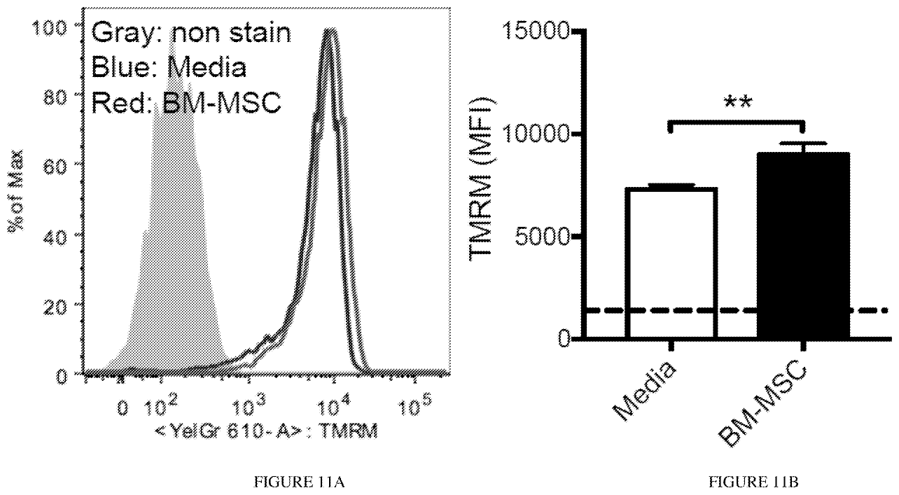

FIGS. 11A-11B show TGF-β induced UCB iTreg were harvested during IL-2 driven expansion in either media/IL-2 alone (media) v. media/IL-2 over BM- MSC (BM-MSC) and surface stained with CD4-APC. Cells were resuspended in media at concentration 2×10 e6 cells/ml. Cells were incubated 30 min at 37° C. with Tetramethylrhodamine, methyl ester (TMRM) (20 nM). Cells were washed with buffer and analyzed. Data shown from two different experiments. **, P<0.01. This data demonstrates that iTregs expanded ex vivo in IL-2/media over a MSC monolayer have increased mitochondrial membrane potential. These results support that MSC mitochondria enhance iTregs function by enhanced mitochondrial activity after transfer.

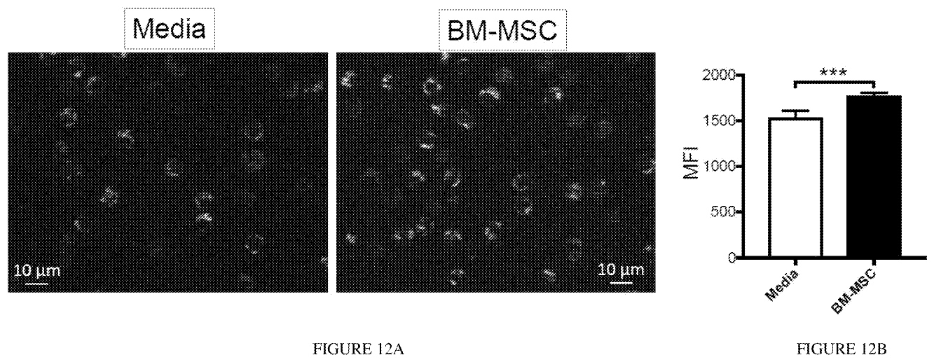

FIGS. 12A-12B show iTregs ex vivo expanded over BM-MSC have increased mitochondria membrane potential. These results support that BM-MSC mitochondria enhance iTregs function by increased mitochondria activity after transfer into iTregs. Analysis of recorded images was performed using ImageJ or Zen 2012 (Carl Zeiss) software. Data shown from two different experiments. ***, P<0.01.

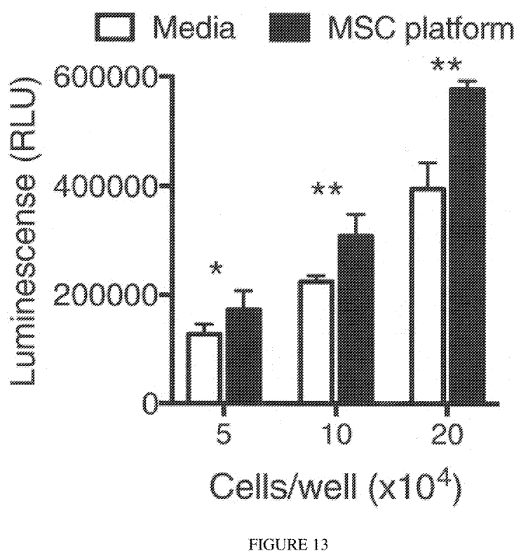

FIG. 13 shows iTreg's ATP were enhanced in BM MSC platform.

Additional experiments were conducted to determine the mechanisms that drive MSC mitochondrial transfer into proliferating iTregs. Treg express apyrases (CD39) and ecto-5′-nucleotidase (CD73) which have been shown to contribute to their inhibitory function by generating adenosine (Alam et al., 2009; Kerkela et al., 2016). Also, it has been shown that CD73-generated adenosine induces cortical actin polymerization via adenosine Al receptor (A1R) induction of a Rho GTPase CDC42-dependent conformational change of the actin-related proteins 2 and 3 (ARP2/3) actin polymerization complex member N-WASP (Bowser et al., 2016). To test whether CD73 contributes to drive MSC mitochondrial transfer, CD73 blocking Ab was added to MSC co-culture and iTreg mitochondrial mass was measured.

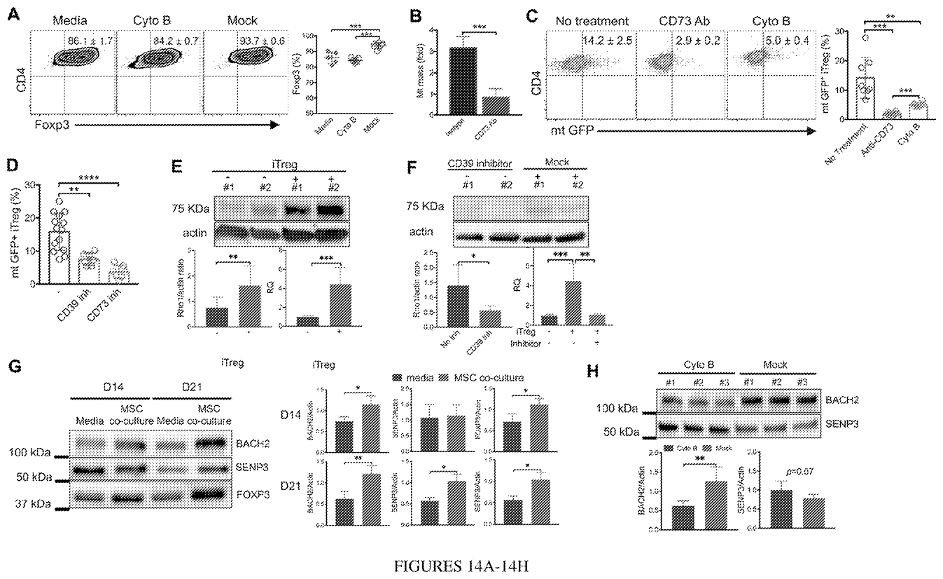

FIG. 14B shows that after CD 73 blocking, iTreg mitochondrial mass was significantly diminished.

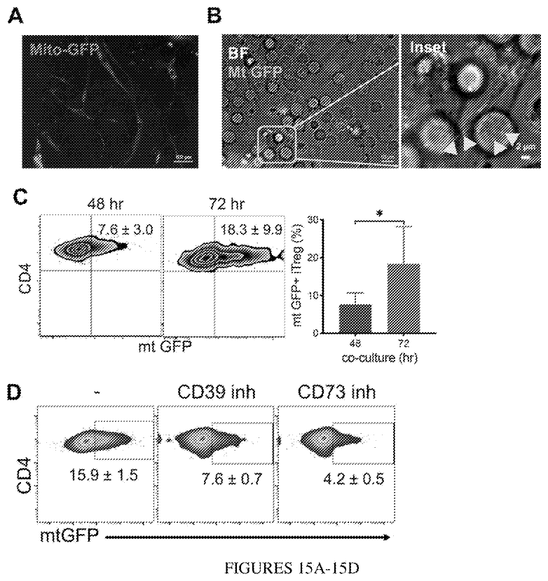

FIGS. 14A-14H and 15A-15D show that the CD39/CD73 pathway drives MSC mitochondrial (mt) transfer into proliferating iTreg. MSCs were transduced with mt-GFP lentivirus to generate stable mt-GFP+MSCs (FIG. 15A). Mt-GFP+iTregs were detected and were significantly increased during 21 day co-cultured with mt-GFP lentivirus transduced MSCs (FIGS. 15B-15C). Mt-GFP+iTregs were significantly decreased with surface CD73 blocking or inhibition of TNT formation (FIG. 14C). As CD39 and CD73calibrate purinergic signals delivered to immune cells through the conversion of ADP/ATP to AMP and AMP to adenosine, respectively. (Allard et al., 2017; Antonioli et al., 2013). Experiments were conducted to inhibit each pathway and noted significantly reduced mt-GFP+iTregs (FIGS. 14D and 15D).

As Mirol has been shown to be a key regulator in mitochondrial intracellular transport (Liu et al., 2012), experiments were designed to determine its role, if any, in MSC mitochondrial transfer to iTreg via TNT. Mirol expression was measured in MSC after co-culture with iTregs. iTreg co-cultivation was associated with significantly enhanced expression of Mirol in MSC including both protein and RNA levels (FIG. 14E). Further, the enhanced MSC expression of Mirol was inhibited by CD39 inhibition (FIG. 14F).

BACH2 has been shown to maintain the stability and function of murine Treg (Kim et al., 2014; Roychoudhuri et al., 2013). BACH2 was previously identified as highly expressed in human iTreg and plays a key role in iTreg stability (Do et al., 2018). Additional studies have identified a pathway by which SENP3 modulates the SUMOylation of BACH2 to control iTreg stability in response to changes in environmental conditions, particularly intracellular ROS (Yu et al., 2018). It was postulated that mitochondrial transfer from MSC may modulate BACH2/SENP3 expression and/or function, given the previous observations of MSC mitochondrial transfer exerting a positive effect on iTreg Foxp3 stability and suppressive function (Jang et al., 2015; Watanabe-Matsui et al., 2011; Yu et al., 2018). iTreg BACH2 and SENP3 protein expression was measured, and MSC co-culture and standard IL-2/media conditions during short-term (day 14 and 21) in vitro culture were compared. iTreg Foxp3, BACH2 and SENP3 expression was notably significantly increased in MSC co-culture conditions (FIG. 14G). The effect of MSC mitochondrial transfer on iTreg BACH2 and SENP3 protein levels after cytochalasin B treatment was examined next. Levels of iTreg BACH2 protein expression were dramatically decreased (FIG. 14H). Cytochalasin B treatment had no significant effect on iTreg SENP3 protein expression, possibly due to SENP3 activation and/or function in this setting (FIG. 14H). Overall, this data suggests that MSC mitochondrial transfer is associated with enhanced BACH2 expression and due to its regulation of Foxp3, positive benefit of enhanced and stable Foxp3 expression.

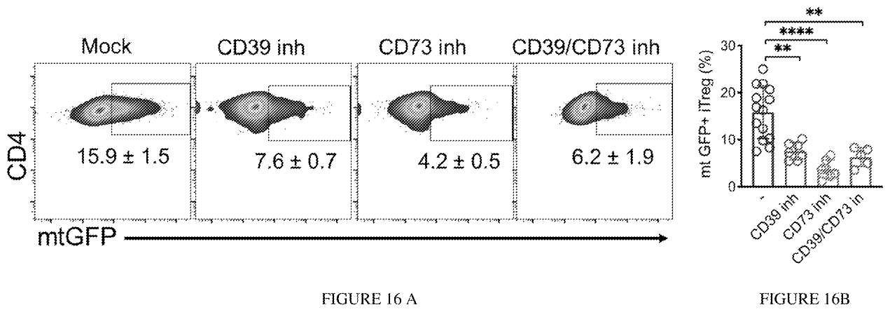

FIGS. 16A-16B show that CD39/CD73 pharmacological inhibitors block transfer of MSC mitochondria into UCB iTregs during IL-2 driven ex vivo expansion. To test whether mitochondrial transfer is mediated by CD39 and CD73 signals, pharmacological inhibitors were added during IL-2 driven expansion. Differentiated Foxp3+ iTreg cells were added to mt-GFP lentiviral transduced MSC platform (24 well plate) at 5×10⁵ cells/ml with IL-2 (100 U/ml) for 72 hrs. To block CD39 and CD73 signal, 50 uM CD39 inhibitor (POM1, a E-NTPDases inhibitor; sigma) and 100 uM CD73 inhibitor (Adenosine 5′-(α,(β-methylene)diphosphate; sigma) or together were added into culture. iTregs were collected at 72 hrs after culture. iTregs were surface stained with anti-human CD4 APC antibody for Flow analysis. Stained cells were analyze with FACS Fortessa . Data are from 3-4 individual experiments (n=5-14). **, p<0.01; ***, p<0.001. These data demonstrate that treatment of CD39/CD73 inhibitor significantly blocks mitochondrial transfer from BM-MSC to UCB iTregs during IL-2 driven ex vivo expansion. Collectively, these results suggest that mitochondrial transfer from BM-MSC to UCB iTregs during IL-2 driven ex vivo expansion is mediated by the ectoenzymes CD39 and CD73 pathway.

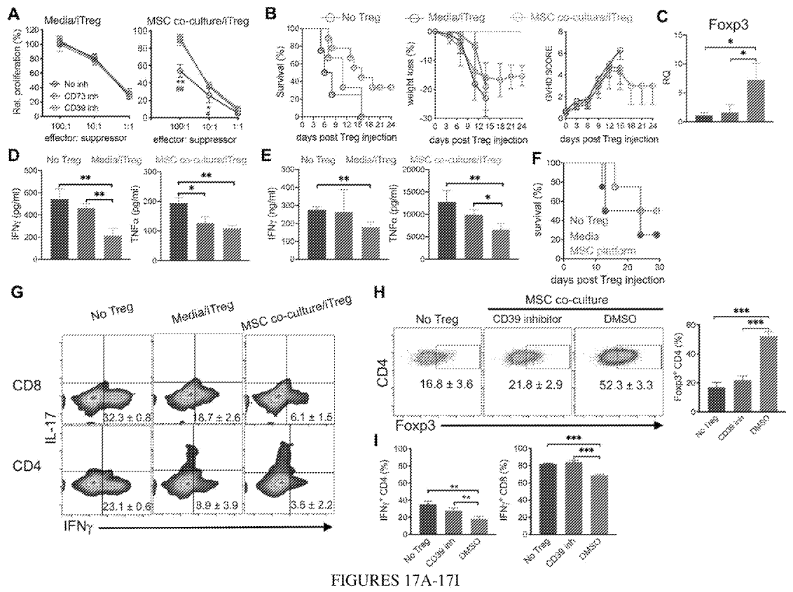

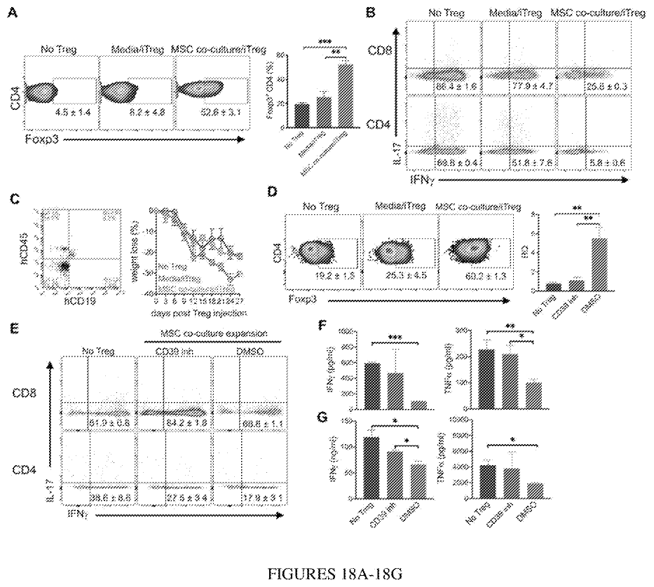

FIGS. 17A-17I and 18A-18G show that MSC co-culture with iTregs ameliorates xenogeneic GVHD and allogeneic GVHD in humanized mouse model. Inhibition of MSC mitochondrial transfer into iTregs resulted in significantly reduced suppressive function vs. control (FIG. 17A). For assessment of the in vivo functions of iTreg in MSC co-culture during iTreg ex vivo expansion in IL-2/media, iTregs were adoptively transferred into GVHD induced NSG mice. This xenogeneic model of GVHD was used in which iTregs culture expanded short-term (21 days) were injected 7 days after adult human PBMC iv injection to induce GVHD. Treatment groups were blinded to technicians performing GVHD (Ehx et al., 2018) and survival assessments (Sonntag et al., 2015).

Mice treated with MSC co-culture iTregs demonstrated significantly improved survival, stable weight, and lower GVHD clinical scores (FIG. 17B). Foxp3 expression and Foxp3+ CD4 T cells in harvested spleen were significantly higher in mice treated with MSC co-cultured iTregs at 2 weeks after GVHD induction (FIGS. 17C and 18A). IFNγ producing CD8+ and CD4+ T cells were dramatically reduced in spleen cells harvested from mice treated with MSC co-cultured iTregs (FIG. 18B). Consistent with cytokine staining, serum and ex vivo levels of pro-inflammatory cytokine IFNγ and TNFα were significantly reduced in animals treated with MSC co-cultured iTregs (FIGS. 17D and 17E).

To determine whether MSC co-culture expanded iTregs have a therapeutic effect in autoimmune disease, a chronic GVHD model using humanized mice which is similar with autoimmune phenotype was used (Sonntag et al., 2015). Human CD45+ engraftment was measured in mice which received CD34+ cells at 24 weeks (FIG. 18C). MSC co-culture expanded iTreg received mice had slightly decreased body weight loss than the other group (FIG. 17C) but survival was improved (FIG. 17F). Foxp3+ Treg cells were significantly enhanced in MSC co-culture expanded iTreg transferred mice (FIG. 18D). It was observed that IFNγ producing CD8+ and CD4+ T cells were significantly reduced in MSC co-culture expanded iTreg treated mice (FIG. 17G). To explore the effect of MSC mitochondrial transfer on iTregs in vivo, MSC co-culture iTregs with added CD39 inhibitor were generated. CD39 inhibitor treatment reduced Foxp3+iTreg percentage in adult PBL-induced GVHD (FIG. 17H). IFNγ producing CD8+ and CD4+ T cells were significantly increased in CD39 inhibitor treated iTreg treated mice (FIGS. 171 and 18E). The level of serum and production of IFNγ and TNFα were significantly increased in CD39 inhibitor iTreg treated mice compared to the control (FIGS. 18F and 18G). Collectively, these results reveal that the suppressive iTregs were maintained by MSC mitochondrial transfer during IL-2 driven ex vivo expansion, and further that these iTregs can protect autoimmune disease in vivo.

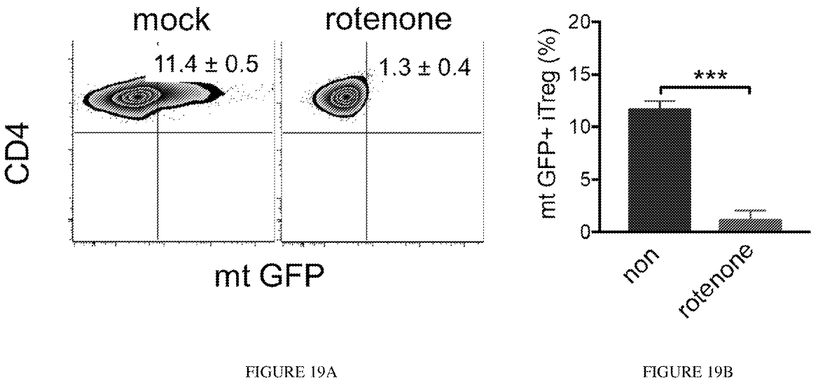

FIGS. 19A-19B show that dysfunctional mitochondria do not transfer into iTregs. To test whether apoptotic MSCs transfer mitochondria into proliferating iTregs, 3×10⁵ MSCs were incubated with mt-GFP lentiviral and seeded into 24 well plate. MSCs were incubated 30 minutes at 37 degrees and cells were washed with media. Foxp3+ iTreg cells were added into mt-GFP lentiviral transduced mock or rotenone treated MSC platform (24 well plate) at 5×10⁵ cells/ml with IL-2 (100 U/ml) in culture for 72 hrs. iTregs were surface stained with anti-human CD4 APC antibody for Flow analysis. Stained cells were analyzed with FACS Fortessa . Data are from two different experiments. N=5-6, *** p<0.001.

Intrinsic defects in Tregs as observed in autoimmune disease patients may hamper the success of autologous nTreg therapies (Kumar et al., 2006; Valencia et al., 2006; Viglietta et al., 2004). Previous studies have shown that ex vivo-expanded, partially HLA-matched nTregs from allogeneic UCB are well tolerated in humans (Brunstein et al., 2011). A major obstacle to the clinical implementation of UCB iTregs is the instability of iTreg Foxp3 expression and loss of suppressor function upon transfer to an inflammatory environment (Hippen et al., 2011). The results reported in this invention demonstrate that BM-MSC co-culture during IL-2 driven ex vivo expansion provides new physiological condition to sustain Foxp3 expression and thereby iTreg suppressive function in inflammatory milieus in vitro and in vivo.

MSC co-culture was observed to be associated with significantly upregulated expression of CD25, CTLA-4, and ICOS, while expression of LAG-3 and TIM-3, which may contribute to exhaustion of activated T cells, was decreased. Higher proportions of CD62L+ and CD45RA± iTreg cells after short-term (21 day) MSC co-culture was observed. Inflammatory environments are attributed to the rapid loss of Foxp3 expression and functional activity of iTregs (Koenecke et al., 2009). The data show a significantly enhanced suppressive function, including inhibition of effector cell activation and maintained Foxp3 stability in MSC co-culture expanded iTregs exposed to inflammatory conditions in vitro and in vivo. It will be important to examine the epigenetic modification of the Treg-specific demethylated region in the Foxp3 gene to gain further insights into the mechanisms of BM-MSC-enhanced iTreg stability (Someya et al., 2017).

Although prior studies point to MSC enhancement of Treg function to be primarily via paracrine mechanisms (Del Fattore et al., 2015), this invention identifies that MSC support of iTreg Foxp3 expression and sustained suppressive function requires direct cell-cell contact. Further, this invention includes the surprising finding that a key mechanism involves mitochondrial transfer by MSC. As the cellular and molecular mechanisms driving MSC mitochondrial transfer to proliferating cells have not been previously been elucidated, this invention further identifies that iTreg CD39/CD73 signaling drives MSC mitochondrial transfer and further that mitochondrial transfer results in augmented iTreg BACH2 and SENP3 expression. BACH2 regulates human UCB iTreg development via direct transcriptional activity at the Foxp3 promoter (Do et al., 2018). SENP3 is a SUMO specific protease that serves to maintain Treg stability (Yu et al., 2018).

CD39 and CD73 play together strategic roles in immune responses (Allard et al., 2017; Antonioli et al., 2013). CD39 and CD73 degrade extracellular ATP to yield AMP and anti-inflammatory adenosine (Deaglio et al., 2007). CD73−/− mice show enhanced antitumor immunity (Stagg et al., 2011) and worse gastritis compared to functional CD73 controls, and adoptive injection of WT Tregs reverses these immune responses (Alam et al., 2009). This invention and others identify (Ehrentraut et al., 2013; Kobie et al., 2006) that CD73 signaling on Tregs is critical to maintain Treg suppressive function. As mitochondrial transfer to Tregs has not been previously examined, this invention identifies that CD39 and CD73 signaling on proliferating iTreg drives MSC mitochondria transfer into iTregs during IL-2 driven ex vivo expansion.

Mirol has been shown to regulate intercellular mitochondrial transfer and enhance injured cell recovery (Ahmad et al., 2014). In a previous study injured astrocytes induced increased levels of Mirol expression in MSC and this was correlated with mitochondrial transfer (Babenko et al., 2018). In the present invention, data show that proliferating iTregs induced Mirol expression on BM-MSC during co-cultivation ex vivo. Further, MSC mitochondrial transfer occurs via TNT.

Mitochondria are an important source of ROS (Turrens, 2003). Interestingly, ROS can regulate cell cycle and function in signaling (Byun et al., 2008; Schieke et al., 2008) and are critical for cancer cell tumorigenicity (Weinberg et al., 2010). This is particularly interesting in light of the finding that ROS is a major driving force for mitochondrial transfer via TNTs from bone marrow stromal cells to leukemic blasts (Marlein et al., 2017). Further work has demonstrated that mitochondrial metabolism also plays a critical role in T cell activation. This invention identifies for the first time the role of CD39/73 expression on proliferating iTreg that drives the transfer of MSC mitochondria. The observations in this invention strongly indicate that increased mitochondria quantity in iTregs is derived from MSCs via TNT transfer but intriguingly, metabolic signaling of the proliferating cells rather than ROS expression appears to be a critical mechanism driving this process.

Regardless of cell type, mitochondria are now recognized to play roles that extend well beyond the production of energy. A central idea emerging from many studies is that T cells undergo changes in cellular metabolism during activation (Akkaya et al., 2018). In contrast to quiescent T cells whose metabolic requirements mainly encompass only cellular trafficking and housekeeping functions, actively proliferating cells must generate additional ATP for functions including the generation of intermediates required in various biosynthesis pathways and signaling molecules for anabolic metabolism. Future work is indicated vaulting from this invention to identify how signaling mechanisms can be modulated to enhance mitochondrial transfer. Importantly, this invention supports that mitochondrial transfer is a key mechanism of MSC supportive function for proliferating cells. Importantly, iTreg mitochondria membrane potential is boosted by co-culture with MSCs. Second, pharmacological inhibition of mitochondrial transfer nearly completely negates the benefit of MSCs on iTreg Foxp3 expression and suppressive function.

In summary, these surprising results highlight the importance of mitochondrial functions in maintaining iTreg FoxP3 expression and suppressive function in inflammatory conditions in vitro and in vivo. Although, several factors can modulate iTreg stability and functions in different disease models, these results provide that the mitochondrial BACH2 pathway is a prominent mechanism of MSC-enhanced iTreg stability and function, and CD39/73 signaling on proliferating cells drives MSC-mediated mitochondrial transfer.

These and additional embodiments of the invention will be apparent to one skilled in the art upon a review of the present disclosure, which is not intended to be limiting to the scope of the claims. The references cited are hereby incorporated by reference herein in their entireties.

### REFERENCES

Ahmad, T., Mukherjee, S., Pattnaik, B., Kumar, M., Singh, S., Kumar, M., Rehman, R., Tiwari, B. K., Jha, K. A., Barhanpurkar, A. P., et al. (2014). Mirol regulates intercellular mitochondrial transport & enhances mesenchymal stem cell rescue efficacy. EMBO J 33, 994-1010.
Akkaya, B., Roesler, A. S., Miozzo, P., Theall, B. P., Al Souz, J., Smelkinson, M. G., Kabat, J., Traba, J., Sack, M. N., Brzostowski, J. A., et al. (2018). Increased Mitochondrial Biogenesis and Reactive Oxygen Species Production Accompany Prolonged CD4(+) T Cell Activation. J Immunol 201, 3294-3306.
Alam, M. S., Kurtz, C. C., Rowlett, R. M., Reuter, B. K., Wiznerowicz, E., Das, S., Linden, J., Crowe, S. E., and Ernst, P. B. (2009). CD73 is expressed by human regulatory T helper cells and suppresses proinflammatory cytokine production and Helicobacter felis-induced gastritis in mice. J Infect Dis 199, 494-504.
Allard, B., Longhi, M. S., Robson, S. C., and Stagg, J. (2017). The ectonucleotidases CD39 and CD73: Novel checkpoint inhibitor targets. Immunol Rev 276, 121-144.
Antonioli, L., Pacher, P., Vizi, E. S., and Hasko, G. (2013). CD39 and CD73 in immunity and inflammation. Trends Mol Med 19, 355-367.
Babenko, V. A., Silachev, D. N., Popkov, V. A., Zorova, L. D., Pevzner, I. B., Plotnikov, E. Y., Sukhikh, G. T., and Zorov, D. B. (2018). Mirol Enhances Mitochondria Transfer from Multipotent Mesenchymal Stem Cells (MMSC) to Neural Cells and Improves the Efficacy of Cell Recovery. Molecules 23.
Babenko, V. A., Silachev, D. N., Zorova, L. D., Pevzner, I. B., Khutornenko, A. A., Plotnikov, E. Y., Sukhikh, G. T., and Zorov, D. B. (2015). Improving the Post-Stroke Therapeutic Potency of Mesenchymal Multipotent Stromal Cells by Cocultivation With Cortical Neurons: The Role of Crosstalk Between Cells. Stem Cells Transl Med 4, 1011-1020.
Baecher-Allan, C., Brown, J. A., Freeman, G. J., and Hafler, D. A. (2001). CD4+CD25high regulatory cells in human peripheral blood. J Immunol 167, 1245-1253.

Beres, A., Komorowski, R., Mihara, M., and Drobyski, W. R. (2011). Instability of Foxp3 expression limits the ability of induced regulatory T cells to mitigate graft versus host disease. Clin Cancer Res 17, 3969-3983.
Bluestone, J. A., Buckner, J. H., Fitch, M., Gitelman, S. E., Gupta, S., Hellerstein, M. K., Herold, K. C., Lares, A., Lee, M. R., Li, K., et al. (2015a). Type 1 diabetes immunotherapy using polyclonal regulatory T cells. Sci Transl Med 7, 315ra189.
Bluestone, J. A., Trotta, E., and Xu, D. (2015b). The therapeutic potential of regulatory T cells for the treatment of autoimmune disease. Expert Opin Ther Targets 19, 1091-1103.
Bowser, J. L., Blackburn, M. R., Shipley, G. L., Molina, J. G., Dunner, K., Jr., and Broaddus, R. R. (2016). Loss of CD73-mediated actin polymerization promotes endometrial tumor progression. J Clin Invest 126, 220-238.
Brunstein, C. G., Miller, J. S., Cao, Q., McKenna, D. H., Hippen, K. L., Curtsinger, J., Defor, T., Levine, B. L., June, C. H., Rubinstein, P., et al. (2011). Infusion of ex vivo expanded T regulatory cells in adults transplanted with umbilical cord blood: safety profile and detection kinetics. Blood 117, 1061-1070.
Byun, H. O., Kim, H. Y., Lim, J. J., Seo, Y. H., and Yoon, G. (2008). Mitochondrial dysfunction by complex II inhibition delays overall cell cycle progression via reactive oxygen species production. Journal of cellular biochemistry 104, 1747-1759.
Chang, K. T., Niescier, R. F., and Min, K. T. (2011). Mitochondrial matrix Ca2+ as an intrinsic signal regulating mitochondrial motility in axons. Proc Natl Acad Sci U S A 108, 15456-15461.
Chen, W., Jin, W., Hardegen, N., Lei, K. J., Li, L., Marinos, N., McGrady, G., and Wahl, S. M. (2003). Conversion of peripheral CD4+ CD25− naive T cells to CD4+ CD25+ regulatory T cells by TGF-beta induction of transcription factor Foxp3. The Journal of experimental medicine 198, 1875-1886.
Davidson, T. S., DiPaolo, R. J., Andersson, J., and Shevach, E. M. (2007). Cutting Edge: IL-2 is essential for TGF-beta-mediated induction of Foxp3+ T regulatory cells. Journal of immunology 178, 4022-4026.
Deaglio, S., Dwyer, K. M., Gao, W., Friedman, D., Usheva, A., Erat, A., Chen, J. F., Enjyoji, K., Linden, J., Oukka, M., et al. (2007). Adenosine generation catalyzed by CD39 and CD73 expressed on regulatory T cells mediates immune suppression. J Exp Med 204, 1257-1265.
Del Fattore, A., Luciano, R., Pascucci, L., Goffredo, B. M., Giorda, E., Scapaticci, M., Fierabracci, A., and Muraca, M. (2015). Immunoregulatory Effects of Mesenchymal Stem Cell-Derived Extracellular Vesicles on T Lymphocytes. Cell Transplant 24, 2615-2627.
Do, J. S., Zhong, F., Huang, A. Y., Van't Hof, W. J., Finney, M., and Laughlin, M. J. (2018). Foxp3 expression in induced T regulatory cells derived from human umbilical cord blood vs. adult peripheral blood. Bone Marrow Transplant 53, 1568-1577.
Du, Y. M., Zhuansun, Y. X., Chen, R., Lin, L., Lin, Y., and Li, J. G. (2018). Mesenchymal stem cell exosomes promote immunosuppression of regulatory T cells in asthma. Exp Cell Res 363, 114-120.
Ehrentraut, H., Clambey, E. T., McNamee, E. N., Brodsky, K. S., Ehrentraut, S. F., Poth, J. M., Riegel, A. K., Westrich, J. A., Colgan, S. P., and Eltzschig, H. K. (2013). CD73+ regulatory T cells contribute to adenosine-mediated resolution of acute lung injury. FASEB J 27, 2207-2219.
Ehx, G., Somja, J., Warnatz, H. J., Ritacco, C., Hannon, M., Delens, L., Fransolet, G., Delvenne, P., Muller, J., Beguin, Y., et al. (2018). Xenogeneic Graft-Versus-Host Disease in Humanized NSG and NSG-HLA-A2/HHD Mice. Front Immunol 9, 1943.
Goodman, W. A., Levine, A. D., Massari, J. V., Sugiyama, H., McCormick, T. S., and Cooper, K. D. (2009). IL-6 signaling in psoriasis prevents immune suppression by regulatory T cells. J Immunol 183, 3170-3176.
Han, H., Hu, J., Yan, Q., Zhu, J., Zhu, Z., Chen, Y., Sun, J., and Zhang, R. (2016). Bone marrow-derived mesenchymal stem cells rescue injured H9c2 cells via transferring intact mitochondria through tunneling nanotubes in an in vitro simulated ischemia/reperfusion model. Molecular medicine reports 13, 1517-1524.
Hayakawa, K., Esposito, E., Wang, X., Terasaki, Y., Liu, Y., Xing, C., Ji, X., and Lo, E.H. (2016). Transfer of mitochondria from astrocytes to neurons after stroke. Nature 535, 551-555.
Hippen, K. L., Merkel, S. C., Schirm, D. K., Nelson, C., Tennis, N. C., Riley, J. L., June, C. H., Miller, J. S., Wagner, J. E., and Blazar, B. R. (2011). Generation and large-scale expansion of human inducible regulatory T cells that suppress graft-versus-host disease. Am J Transplant 11, 1148-1157.
Huehn, J., Polansky, J. K., and Hamann, A. (2009). Epigenetic control of FOXP3 expression: the key to a stable regulatory T-cell lineage? Nat Rev Immunol 9, 83-89.
Jang, K. J., Mano, H., Aoki, K., Hayashi, T., Muto, A., Nambu, Y., Takahashi, K., Itoh, K., Taketani, S., Nutt, S. L., et al. (2015). Mitochondrial function provides instructive signals for activation-induced B-cell fates. Nat Commun 6, 6750.
Kanamori, M., Nakatsukasa, H., Okada, M., Lu, Q., and Yoshimura, A. (2016). Induced Regulatory T Cells: Their Development, Stability, and Applications. Trends in immunology 37, 803-811.
Kasakovski, D., Xu, L., and Li, Y. (2018). T cell senescence and CAR-T cell exhaustion in hematological malignancies. J Hematol Oncol 11, 91.
Kerkela, E., Laitinen, A., Rabina, J., Valkonen, S., Takatalo, M., Larjo, A., Veijola, J., Lampinen, M., Siljander, P., Lehenkari, P., et al. (2016). Adenosinergic Immunosuppression by Human Mesenchymal Stromal Cells Requires Co-Operation with T cells. Stem Cells 34, 781-790.
Kim, E. H., Gasper, D. J., Lee, S. H., Plisch, E. H., Svaren, J., and Suresh, M. (2014). Bach2 regulates homeostasis of Foxp3+ regulatory T cells and protects against fatal lung disease in mice. J Immunol 192, 985-995.
Kobie, J. J., Shah, P. R., Yang, L., Rebhahn, J. A., Fowell, D.J., and Mosmann, T. R. (2006). T regulatory and primed uncommitted CD4 T cells express CD73, which suppresses effector CD4 T cells by converting 5′-adenosine monophosphate to adenosine. J Immunol 177, 6780-6786.
Koenecke, C., Czeloth, N., Bubke, A., Schmitz, S., Kissenpfennig, A., Malissen, B., Huehn, J., Ganser, A., Forster, R., and Prinz, I. (2009). Alloantigen-specific de novo-induced Foxp3+ Treg revert in vivo and do not protect from experimental GVHD. Eur J Immunol 39, 3091-3096.
Kumar, M., Putzki, N., Limmroth, V., Remus, R., Lindemann, M., Knop, D., Mueller, N., Hardt, C., Kreuzfelder, E., and Grosse-Wilde, H. (2006). CD4+ CD25+ FoxP3+ T lymphocytes fail to suppress myelin basic protein-induced proliferation in patients with multiple sclerosis. J Neuroimmunol 180, 178-184.
Lange, C., Scholl, M., Melms, A., and Bischof, F. (2011). CD62L(high) Treg cells with superior immunosuppressive properties accumulate within the CNS during remissions of EAE. Brain, behavior, and immunity 25, 120-126.
Le Texier, L., Lineburg, K. E., Cao, B., McDonald-Hyman, C., Leveque-El Mouttie, L., Nicholls, J., Melino, M., Nalkurthi, B. C., Alexander, K. A., Teal, B., et al. (2016). Autophagy-dependent regulatory T cells are critical for the control of graft-versus-host disease. JCI Insight 1, e86850.
Lee, S., Park, K., Kim, J., Min, H., and Seong, R.H. (2018). Foxp3 expression in induced regulatory T cells is stabilized by C/EBP in inflammatory environments. EMBO Rep 19.
Leprat, P., Ratinaud, M. H., and Julien, R. (1990). A new method for testing cell ageing using two mitochondria specific fluorescent probes. Mech Ageing Dev 52, 149-167.
Liu, S., Sawada, T., Lee, S., Yu, W., Silverio, G., Alapatt, P., Millan, I., Shen, A., Saxton, W., Kanao, T., et al. (2012). Parkinson's disease-associated kinase PINK1 regulates Miro protein level and axonal transport of mitochondria. PLoS Genet 8, e1002537.
Long, S. A., Cerosaletti, K., Bollyky, P. L., Tatum, M., Shilling, H., Zhang, S., Zhang, Z. Y., Pihoker, C., Sanda, S., Greenbaum, C., and Buckner, J. H. (2010). Defects in IL-2R signaling contribute to diminished maintenance of FOXP3 expression in CD4(+)CD25(+) regulatory T-cells of type 1 diabetic subjects. Diabetes 59, 407-415.

Lu, S. Y., Liu, K. Y., Liu, D. H., Xu, L. P., and Huang, X. J. (2011). High frequencies of CD62L(+) naive regulatory T cells in allografts are associated with a low risk of acute graft-versus-host disease following unmanipulated allogeneic haematopoietic stem cell transplantation. Clinical and experimental immunology 165, 264-277.
Marlein, C. R., Zaitseva, L., Piddock, R. E., Robinson, S. D., Edwards, D. R., Shafat, M. S., Zhou, Z., Lawes, M., Bowles, K. M., and Rushworth, S. A. (2017). NADPH oxidase-2 derived superoxide drives mitochondrial transfer from bone marrow stromal cells to leukemic blasts. Blood 130, 1649-1660.
Melief, S. M., Schrama, E., Brugman, M. H., Tiemessen, M. M., Hoogduijn, M. J., Fibbe, W. E., and Roelofs, H. (2013). Multipotent stromal cells induce human regulatory T cells through a novel pathway involving skewing of monocytes toward anti-inflammatory macrophages. Stem Cells 31, 1980-1991.
Quintero, O. A., DiVito, M. M., Adikes, R. C., Kortan, M. B., Case, L. B., Lier, A. J., Panaretos, N. S., Slater, S. Q., Rengarajan, M., Feliu, M., and Cheney, R. E. (2009). Human Myo19 is a novel myosin that associates with mitochondria. Curr Biol 19, 2008-2013.
Rauch, K. S., Hils, M., Lupar, E., Minguet, S., Sigvardsson, M., Rottenberg, M. E., Izcue, A., Schachtrup, C., and Schachtrup, K. (2016). Id3 Maintains Foxp3 Expression in Regulatory T Cells by Controlling a Transcriptional Network of E47, Spi-B, and SOCS3. Cell Rep 17, 2827-2836.
Rojewski, M. T., Weber, B. M., and Schrezenmeier, H. (2008). Phenotypic Characterization of Mesenchymal Stem Cells from Various Tissues. Transfus Med Hemother 35, 168-184.
Roychoudhuri, R., Eil, R. L., Clever, D., Klebanoff, C. A., Sukumar, M., Grant, F. M., Yu, Z., Mehta, G., Liu, H., Jin, P., et al. (2016). The transcription factor BACH2 promotes tumor immunosuppression. J Clin Invest 126, 599-604.
Roychoudhuri, R., Hirahara, K., Mousavi, K., Clever, D., Klebanoff, C. A., Bonelli, M., Sciume, G., Zare, H., Vahedi, G., Dema, B., et al. (2013). BACH2 represses effector programs to stabilize T(reg)-mediated immune homeostasis. Nature 498, 506-510.
Schieke, S. M., McCoy, J. P., Jr., and Finkel, T. (2008). Coordination of mitochondrial bioenergetics with G1 phase cell cycle progression. Cell cycle 7, 1782-1787.
Schmitt, E. G., and Williams, C. B. (2013). Generation and function of induced regulatory T cells. Frontiers in immunology 4, 152.
Sinclair, K., Yerkovich, S. T., and Chambers, D. C. (2013). Mesenchymal stem cells and the lung. Respirology 18, 397-411.
Someya, K., Nakatsukasa, H., Ito, M., Kondo, T., Tateda, K. I., Akanuma, T., Koya, I., Sanosaka, T., Kohyama, J., Tsukada, Y. I., et al. (2017). Improvement of Foxp3 stability through CNS2 demethylation by TET enzyme induction and activation. Int Immunol 29, 365-375.
Sonntag, K., Eckert, F., Welker, C., Muller, H., Muller, F., Zips, D., Sipos, B., Klein, R., Blank, G., Feuchtinger, T., et al. (2015). Chronic graft-versus-host-disease in CD34(+)-humanized NSG mice is associated with human susceptibility HLA haplotypes for autoimmune disease. J Autoimmun 62, 55-66.
Stagg, J., Divisekera, U., Duret, H., Sparwasser, T., Teng, M. W., Darcy, P. K., and Smyth, M. J. (2011). CD73-deficient mice have increased antitumor immunity and are resistant to experimental metastasis. Cancer Res 71, 2892-2900.
Tasso, R., Ilengo, C., Quarto, R., Cancedda, R., Caspi, R. R., and Pennesi, G. (2012). Mesenchymal stem cells induce functionally active T-regulatory lymphocytes in a paracrine fashion and ameliorate experimental autoimmune uveitis. Investigative ophthalmology & visual science 53, 786-793.
Tone, Y., Furuuchi, K., Kojima, Y., Tykocinski, M. L., Greene, M. I., and Tone, M. (2008). Smad3 and NFAT cooperate to induce Foxp3 expression through its enhancer. Nat Immunol 9, 194-202.
Trzonkowski, P., Szarynska, M., Mysliwska, J., and Mysliwski, A. (2009). Ex vivo expansion of CD4(+)CD25(+) T regulatory cells for immunosuppressive therapy. Cytometry A 75, 175-188.
Turrens, J. F. (2003). Mitochondrial formation of reactive oxygen species. The Journal of physiology 552, 335-344.
Valencia, X., Stephens, G., Goldbach-Mansky, R., Wilson, M., Shevach, E. M., and Lipsky, P. E. (2006). TNF downmodulates the function of human CD4+ CD25hi T-regulatory cells. Blood 108, 253-261.
van Leeuwen, M. A., du Pre, M. F., van Wanrooij, R. L., de Ruiter, L. F., Raatgeep, H. R., Lindenbergh-Kortleve, D. J., Mulder, C. J., de Ridder, L., Escher, J. C., and Samsom, J. N. (2013). Changes in natural Foxp3(+)Treg but not mucosally-imprinted CD62L(neg)CD38(+)Foxp3(+)Treg in the circulation of celiac disease patients. PloS one 8, e68432.
Viglietta, V., Baecher-Allan, C., Weiner, H. L., and Hafler, D. A. (2004). Loss of functional suppression by CD4+ CD25+ regulatory T cells in patients with multiple sclerosis. J Exp Med 199, 971-979.
Vignais, M. L., Caicedo, A., Brondello, J. M., and Jorgensen, C. (2017). Cell Connections by Tunneling Nanotubes: Effects of Mitochondrial Trafficking on Target Cell Metabolism, Homeostasis, and Response to Therapy. Stem Cells Int 2017, 6917941.
Wang, J., Liu, X., Qiu, Y., Shi, Y., Cai, J., Wang, B., Wei, X., Ke, Q., Sui, X., Wang, Y., et al. (2018). Cell adhesion-mediated mitochondria transfer contributes to mesenchymal stem cell-induced chemoresistance on T cell acute lymphoblastic leukemia cells. J Hematol Oncol 11, 11.
Watanabe-Matsui, M., Muto, A., Matsui, T., Itoh-Nakadai, A., Nakajima, 0., Murayama, K., Yamamoto, M., Ikeda-Saito, M., and Igarashi, K. (2011). Heme regulates B-cell differentiation, antibody class switch, and heme oxygenase-1 expression in B cells as a ligand of Bach2. Blood 117, 5438-5448.
Weinberg, F., Hamanaka, R., Wheaton, W. W., Weinberg, S., Joseph, J., Lopez, M., Kalyanaraman, B., Mutlu, G. M., Budinger, G. R., and Chandel, N. S. (2010). Mitochondrial metabolism and ROS generation are essential for Kras-mediated tumorigenicity. Proceedings of the National Academy of Sciences of the United States of America 107, 8788-8793.
Xu, Y., Melo-Cardenas, J., Zhang, Y., Gau, I., Wei, J., Montauti, E., Zhang, Y., Gao, B., Jin, H., Sun, Z., et al. (2019). The E3 ligase Hrd1 stabilizes Tregs by antagonizing inflammatory cytokine-induced ER stress response. JCI Insight 4.
Yu, X., Lao, Y., Teng, X. L., Li, S., Zhou, Y., Wang, F., Guo, X., Deng, S., Chang, Y., Wu, X., et al. (2018). SENP3 maintains the stability and function of regulatory T cells via BACH2 deSUMOylation. Nature communications 9, 3157.
Zheng, J., Chan, P. L., Liu, Y., Qin, G., Xiang, Z., Lam, K. T., Lewis, D. B., Lau, Y. L., and Tu, W. (2013). ICOS regulates the generation and function of human CD4+ Treg in a CTLA-4 dependent manner. PLoS One 8, e82203.
Zorn, E., Nelson, E. A., Mohseni, M., Porcheray, F., Kim, H., Litsa, D., Bellucci, R., Raderschall, E., Canning, C., Soiffer, R. J., et al. (2006). IL-2 regulates FOXP3 expression in human CD4+ CD25+ regulatory T cells through a STAT-dependent mechanism and induces the expansion of these cells in vivo. Blood 108, 1571-1579.
Bao, R., Shui, X., et al. (2016). Adenosine and the adenosine A2A receptor agonist, CGS21680, upregulate CD39 and CD73 expression through E2F-1 and CREB in regulatory T cells isolated from septic mice. Intl J. of Molecular Med., 38, 969-975, DOI: 10.3892/ijmm.2016.2679.
Beavis, P. A., Stagg, J., Darcy, P. K., and Smyth, M. J. (2012). Trends in Immunology, Vol. 33, No. 5.
Chalmin, F., Mignot, G., Bruchard, M., et al. (2012). Immunity 36, 362-373, DOI 10.1016/j.immuni.2011.12.019.
Eltzschig, H. K., Köhler, D., Eckle, T., et al. (2009). Central role of Sp1-regulated CD39 in hypoxia/ischemia protection. Blood, 113, 224-232.
Eltzschig, H. K., Ibla, J. C., Furuta, G. T., et al. (2003). Coordinated Adenine Nucleotide Phosphohydrolysis and Nucleoside Signaling in Posthypoxic Endothelium: Role of Ectonucleotidases and Adenosine A2B Receptors. J of Exp. Med., Vol. 198, No. 5, 783-796 http://doi.org/10.1084/jem.20030891.
Hanna S. J., McCoy-Simandle K., Miskolci V., et al. (2017). The Role of Rho-GTPases and actin polymerization during Macrophage Tunneling Nanotube Biogenesis. Scientific Reports, 7:8547, DOI:10.1038/s41598-017-08950-7 1.
Keller, K. E., Bradley J. M., Sun Y. Y., et al. (2017). Tunneling Nanotubes are Novel Cellular Structures That Communicate Signals Between Trabecular Meshwork Cells. Invest Ophthalmol Vis Sci. 2017;58:5298-5307. DOI:10.1167/iovs.17-22732.
Regateiro, F. S., Howie, D., Nolan, K. F., et al. (2011). Generation of anti-inflammatory adenosine by leukocytes is regulated by TGF-β. Eur. J. Immunol. 2011. 41: 2955-2965, DOI 10.1002/eji.201141512.
Sáenz-de-Santa-Maria, I., Bernardo-Castiñeira, C., Enciso, E., et al. (2017). Control of long-distance cell-to-cell communication and autophagosome transfer in squamous cell carcinoma via tunneling nanotubes. Oncotarget, Vol. 8, No. 13, 20939-20960.

Synnestvedt, K., et al. (2002). Ecto-5′-nucleotidase (CD73) regulation by hypoxia-inducible factor-1 mediates permeability changes in intestinal epithelia. J. Clin Invest. 110(7), 993-1002, https://doi.org/10.1172/JCI15337.

1. A method for producing inducible regulatory T cells (iTregs) from blood comprising:
providing blood;
isolating naïve CD4⁺ T cells from the blood;
inducing the naïve CD4⁺ T cells to differentiate into a first composition comprising iTregs;
separating the iTregs from the first composition to form a substantially purified iTreg composition;
expanding the purified iTreg composition over a mesenchymal stromal cell (MSC) feeder layer; and
inducing tunneling nanotubule (TNT) formation in the MSC feeder layer for increased mitochondrial transfer, to produce an expanded iTreg composition with sustained FoxP3 expression and suppressive function in inflammatory conditions.

2. The method of claim 1, wherein the blood is human umbilical cord blood.

3. The method of claim 1, wherein the inducing step comprises treating the naïve CD4+ T cells with TGF-β.

4. The method of claim 1, wherein the iTregs are separated from the first composition using flow cytometry cell sorting or magnetic cell sorting.

5. The method of claim 1, wherein the purified iTreg composition is at least 90% pure.

6. The method of claim 1, wherein the iTregs express CD4⁺, CD25⁺, and FoxP3⁺ proteins.

7. The method of claim 1, further comprising expanding the purified iTreg composition by increasing BACH2 transcriptional regulation of FoxP3 expression.

8. The method of claim 1, wherein the mitochondrial transfer is promoted by upregulating the CD39 and/or CD 73 pathways on proliferating iTreg.

9. An inducible regulatory T cell composition comprising the expanded iTreg composition produced by claim 1.

10. A method for treating an inflammatory or an autoimmune condition in a human subject in need thereof comprising:
administering to the subject a composition comprising a therapeutically effective dose of umbilical cord blood derived iTregs expanded over mesenchymal stromal cells with induced TNT formation.

11. The method of claim 10, wherein the umbilical cord blood iTregs have been differentiated by inducing BACH2 transcriptional regulation of FoxP3 expression.

12. The method of claim 10, wherein the iTregs are autologous.

13. The method of claim 10, wherein the iTregs are allogeneic.

14. The method of claim 10, wherein the iTregs are specific for a single antigen.

15. The method of claim 10, wherein the iTregs are polyclonal.

16. The method of claim 10, wherein the subject is suffering diabetes complications.

17. A therapeutic regulatory T cell composition comprising an effective dose of umbilical cord blood derived iTregs expanded over mesenchymal stromal cells with induced TNT formation.

18. The composition of claim 17, wherein the umbilical cord blood iTregs have been differentiated by inducing BACH2 transcriptional regulation of FoxP3 expression.

19. A method for treating an immune-related disease or condition in a subject in need thereof comprising administering to the subject an effective amount of the composition of claim 17.

20. The method of claim 19, wherein the therapeutic T cell composition comprises inducible regulatory T cells.

21. The method of claim 19, wherein the therapeutic T cell composition comprises chimeric antigen receptor-expressing T cells.

22. The method of claim 19, wherein the therapeutic T cell composition comprises virus specific effector T cells.

23. The method of claim 19, wherein the subject is suffering from cancer.

24-42. (canceled)
